(19) United States
(12) Patent Application Publication
(10) Pub. No.: US 2023/0002712 A1
(43) Pub. Date: Jan. 5, 2023

(54) Microwell Perfusion Plates for Organoids and Related Systems and Methods

(71) Applicant: Wake Forest University Health Sciences, Winston-Salem, NC (US)

(72) Inventor: Amish Asthana, Whitsett, NC (US)

(21) Appl. No.: 17/779,241

(22) PCT Filed: Nov. 24, 2020
(86) PCT No.: PCT/US2020/061914

§ 371 Date: May 24, 2022

Related U.S. Application Data
(60) Provisional application No. 62/939,799, filed on Nov. 25, 2019.

(51) Int. Cl. C12M 1/32; C12M 1/12; C12M 1/00

(52) U.S. Cl. CPC C12M 23/12; C12M 25/04; C12M 29/10

(57) ABSTRACT

A microwell perfusion plate system includes a plate and at least one well on the plate. Each well includes: a porous membrane; a through-pore microwell membrane above and on the porous membrane, the microwell membrane including a plurality of microwells with a respective microwell configured to hold a 3D cell culture, wherein a respective microwell includes a top opening and a bottom opening; an inlet passageway in fluid communication with each top opening of the plurality of microwells and configured to deliver liquid medium to the plurality of microwells and the 3D cell cultures held therein; an outlet passageway in fluid communication with each bottom opening of the plurality of microwells and configured to receive the liquid medium from the plurality of microwells; and a cell culture well directly above the microwell membrane, wherein the cell culture well defines at least a portion of the inlet passageway.

### RELATED APPLICATIONS

This application claims priority from U.S. Provisional Application Ser. No. 62/939,799, filed Nov. 25, 2019, the disclose of which is incorporated by reference herein in its entirety.

### BACKGROUND

When cells are transferred to non-adherent growth platforms they form clusters or microtissues. However, as there is no physical constraint on size and separation, the microtissues can grow over time or agglomerate, leading to diffusion limitations. Moreover, there is heterogeneity in the size distribution of the microtissues, which can lead to biases.

Microwell arrays that physically restrict spheroid size can be used for achieving a defined and homogenous size. They can also be incorporated in microfluidic devices to sequester spheroids in individual microwells, provide control over fluid flow and test them in a dynamic manner. However, there are several disadvantages associated with conventional microfluidic devices. Medium flow across the face of microwell (tangential) can be inadequate in providing complete medium exchange and removal of debris, especially for deeper microwells. Permanent bonding of devices can make cell loading and recovery of microtissues for downstream analysis difficult. Fabrication requires cleanrooms for soft lithography, and the fabrication material (polydimethylsiloxane; PDMS) is unsuitable due to absorption of hydrophobic reagents and leaching of small molecules. The devices do not have a standard footprint, therefore integration with imaging platforms can be challenging. Finally, such devices might be suited for testing but cannot be easily scaled up for biomanufacturing of stem cell-derived organoids in physiomimetic conditions, discouraging their widespread adoption.

Therefore, there is a need for an open, long-term culture system that provides robust control on the fluidic, biophysical and biochemical microenvironment and allows for multiple characterization and functional readouts in order to optimize critical factors that positively affect the differentiation, maturation, and function of organoids.

### SUMMARY

Some embodiments of the present invention are directed to a microwell perfusion plate system. The system includes a plate and at least one well on the plate. Each well includes a porous membrane and a through-pore microwell membrane having a top and a bottom with the bottom above and on the porous membrane. The microwell membrane includes a plurality of microwells with a respective microwell configured to hold a 3D cell culture. A respective microwell includes a top opening at the top of the microwell membrane and a bottom opening at the bottom of the microwell membrane. Each well includes: an inlet passageway in fluid communication with each top opening of the plurality of microwells and configured to deliver liquid medium to the plurality of microwells and the 3D cell cultures held therein; an outlet passageway in fluid communication with each bottom opening of the plurality of microwells and configured to receive the liquid medium from the plurality of microwells; and a cell culture well directly above the microwell membrane, wherein the cell culture well defines at least a portion of the inlet passageway.

In some embodiments, each well includes a bottom outlet channel below the porous membrane and extending between a central portion of the well and an outer peripheral portion of the well, and wherein the bottom outlet channel defines at least a portion of the outlet passageway. The bottom outlet channel may widen from the central portion of the well to the outer peripheral portion of the well. The bottom outlet channel may have a constant width or narrow from the central portion of the well to the outer peripheral portion of the well. The bottom outlet channel may be defined in the plate.

In some embodiments, each well includes a body comprising at least one layer that is on the microwell membrane and/or the plate. The cell culture well may be defined in a central portion of the body. The body may be bonded to the plate. The body and/or the plate may include PMMA. The body may be on a first side of the plate, and each well may further include a glass coverslip on a second, opposite side of the plate below the bottom outlet channel.

In some embodiments, an outlet medium reservoir is optionally defined in an outer peripheral portion the body. The outlet medium reservoir may be in fluid communication with and positioned above the bottom outlet channel optionally at the outer peripheral portion of the well, wherein the outlet medium reservoir may define at least a portion of the outlet passageway. The outlet medium reservoir may be arcuate and may extend along a portion of the outer peripheral portion the body.

In some embodiments, an inlet medium compartment is defined in the body. The inlet medium compartment may be in fluid communication with and positioned above the cell culture well, wherein the inlet medium compartment may define at least a portion of the inlet passageway. The outlet medium reservoir may be at a first side of the outer peripheral portion of the body. The inlet medium compartment may extend between the outlet medium reservoir and a second, opposite side of the outer peripheral portion of the body.

In some embodiments, an inlet port member at the outer peripheral portion of the body. The inlet port member may include an inlet port configured to receive a pipette tip such that the liquid medium is delivered to the inlet medium compartment.

In some embodiments, the body includes first and second layers. The cell culture well and a lower portion of the outer medium reservoir may be defined in the first layer. The inlet medium compartment and an intermediate or upper portion of the outlet medium reservoir may be defined in the second layer. The inlet port member may be on the second layer.

In some embodiments, the intermediate or upper portion of the outlet medium reservoir is an intermediate portion of the outlet medium reservoir. The body may further include an upper portion of the outlet medium reservoir on the second layer and above the intermediate portion of the outlet medium reservoir.

In some embodiments, the inlet medium compartment diverges into first and second inlet fluid pathways at the outer peripheral portion of the body and the first and second inlet fluid pathways converge at the central portion of the body above the cell culture well.

In some embodiments, the body further includes an inlet and outlet port member comprising an inlet port in fluid communication with and positioned above the inlet medium compartment and an outlet port in fluid communication with and positioned above the outlet medium reservoir.

In some embodiments, the body comprises first, second, and third layers. The cell culture well and a lower portion of the outlet medium reservoir may be defined in the first layer. The inlet medium channel and an upper portion of the outlet medium reservoir may be defined in the second layer. The inlet port and the outlet port may be defined in the third layer.

In some embodiments, the body is monolithic.

In some embodiments, a lid is configured to be selectively installed over the second side of the plate. The lid may include an inlet port and an outlet port for each well. The inlet port of the lid may be in fluid communication with the inlet port of the body and the outlet port of the lid may be in fluid communication with the outlet port of the body. An inlet coupler may be in the inlet port of the lid. An outlet coupler may be in the outlet port of the lid. An inlet tube may be connected to the inlet coupler at a first end of the inlet tube. An outlet tube may be connected to the outlet coupler at a first end of the outlet tube. At least one pump may be provided with a second, opposite end of the inlet tube connected to the at least pump and a second, opposite end of the outlet tube connected to the at least one pump. The at least one pump may be configured to deliver medium to the body through the inlet tube and remove medium from the body through the outlet tube. The inlet coupler may extend downwardly into the inlet port of the inlet and outlet port member. The outlet coupler may extend downwardly into the outlet port of the inlet and outlet port member.

In some embodiments, an insert is configured to be selectively installed in a container held in a respective well. The porous membrane and the through-pore microwell membrane may be on the insert. The cell culture well may be on the insert and may surround the porous membrane and the through-pore microwell membrane. The container may define at least a portion of the outlet passageway.

In some embodiments, a lid is configured to be selectively installed over the first side of the plate. The lid may include an inlet port and an outlet port for each well. The inlet port of the lid may be in fluid communication with the cell culture well and the outlet port of the lid may be in fluid communication with the container. An inlet coupler may be in the inlet port of the lid. An outlet coupler may be in the outlet port of the lid. An inlet tube may be connected to the inlet coupler at a first end of the inlet tube. An outlet tube may be connected to the outlet coupler at a first end of the outlet tube. At least one pump may be provided with a second, opposite end of the inlet tube connected to the at least pump and a second, opposite end of the outlet tube connected to the at least one pump. The pump may be configured to deliver medium to the cell culture well through the inlet tube and remove medium from the container through the outlet tube. The inlet coupler may extend downwardly into the cell culture well and the outlet coupler may extend downwardly into the container.

In some embodiments, the at least one well includes a plurality of wells.

In some embodiments, a respective microwell of the microwell membrane has a pyramidal shape.

In some embodiments, a respective microwell of the microwell membrane includes sloped trapezoidal sidewalls and the top and bottom openings are square.

In some embodiments, a respective microwell of the microwell membrane includes a curved sidewall, the top and bottom openings are circular and/or round, and the microwell has a hemispherical shape.

Some other embodiments of the present invention are directed to a method of culturing cells and/or preparing organoids, spheroids, microtissues, and/or cell clusters. The method includes: providing any of the systems as described above; and perfusing the liquid medium directly through the top opening, past and/or through the 3D cell culture, and then through the bottom opening of each microwell of the microwell membrane.

Some other embodiments of the present invention are directed to a method of culturing cells and/or preparing organoids. The method includes providing a system including: a plate including a plurality of wells; a porous membrane in each well; a microwell through-pore membrane directly above and on the porous membrane, the microwell membrane including a plurality of microwells, each microwell including at least one sidewall defining a top opening at a top of the microwell membrane and a bottom opening at a bottom of the microwell membrane, each microwell configured to hold a 3D cell culture; a cell culture well directly above the microwell membrane; and an outlet medium reservoir with at least a portion of the outlet medium reservoir directly below the porous membrane. The method includes directly perfusing liquid medium through the cell culture well, then through the top opening of each microwell, then past and/or through the 3D cell culture, then through the bottom opening of each microwell, and then through the outlet medium reservoir.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIG. 1 is a general schematic of a microwell perfusion plate system according to some embodiments.

FIGS. 2 and 3 include digital images of a microwell through-pore membrane according to some embodiments.

FIG. 4 includes digital images of the microwell through-pore membrane bonded to a polycarbonate porous membrane according to some embodiments.

FIG. 5 is an exploded view of a body and a microwell through-pore membrane bonded to a polycarbonate porous membrane used in a static microwell perfusion plate system according to some embodiments.

FIG. 6 is an assembled view of the body and microwell through-pore membrane bonded to a polycarbonate porous membrane of FIG. 5.

FIG. 7 is a fragmentary sectional view of the microwell through-pore membrane bonded to a polycarbonate porous membrane of FIG. 5.

FIG. 8 is the body and microwell through-pore membrane bonded to a polycarbonate porous membrane of FIG. 6 with liquid medium therein.

FIG. 9 is a bottom view of the static microwell perfusion plate system using a plate and the components illustrated in FIG. 5.

FIG. 10 is an exploded view of a body and microwell through-pore membrane bonded to a polycarbonate porous membrane used in a first dynamic microwell perfusion plate system according to some other embodiments.

FIG. 11 is an assembled view of the body and microwell through-pore membrane bonded to a polycarbonate porous membrane of FIG. 10.

FIG. 12 is the body and microwell through-pore membrane bonded to a polycarbonate porous membrane of FIG. 11 with liquid medium therein.

FIG. 13A is a bottom view of the first dynamic microwell perfusion plate system using a plate and the components illustrated in FIG. 10.

FIG. 13B is a top perspective view of a lid that can be selectively installed on the system of FIG. 13A.

FIG. 14 is a fragmentary perspective view of a second dynamic microwell perfusion plate system according to some other embodiments.

FIG. 15 is a top perspective view of an insert used with the system of FIG. 14.

FIG. 16 illustrates the insert installed in a container of the system of FIG. 14.

FIG. 17 the insert installed in the container of the system of FIG. 16 with medium in the container and/or insert.

FIG. 18 is a top perspective view of a lid that can be selectively installed on the system of FIG. 14.

FIGS. 19-22 include digital images illustrating alternative microwell configurations.

FIG. 23 is an exploded view of a body and a microwell through-pore membrane bonded to a polycarbonate porous membrane used in a static microwell perfusion plate system according to some embodiments.

FIG. 24 is an assembled view of the body and microwell through-pore membrane bonded to a polycarbonate porous membrane of FIG. 23.

FIG. 25 is the body and microwell through-pore membrane bonded to a polycarbonate porous membrane of FIG. 6 with liquid medium therein.

FIG. 26 is a fragmentary view of a static microwell perfusion plate system using a plate and the components illustrated in FIG. 23.

FIGS. 27A and 27B are digital images using the system of FIG. 26.

FIG. 28 is an assembled view of an alternative embodiment of a body for the first dynamic microwell perfusion plate system of FIGS. 10-13.

### DETAILED DESCRIPTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

“Cells” and “cell” as used in the present invention are, in general, animal cells, particularly mammalian and/or primate cells, examples of which include, but are not limited to human, dog, cat, rabbit, monkey, chimpanzee, cow, pig, and goat. The cells may be differentiated at least in part to a particular cell or tissue type, such as liver, intestine, pancreas, lymph node, smooth muscle, skeletal muscle, cardiac muscle, central nerve, peripheral nerve, skin, bone, lung, breast, testes, immune system, kidney, etc. In some embodiments, the cells are diseased cells, optionally cancer cells. In some embodiments, a cell may express (naturally or by recombinant techniques) a detectable compound, which is a compound that provides and/or generates a detectable signal that allows for differentiation and/or identification of a cell and/or cell population such as, e.g., a fluorescent compound. In some embodiments, cells may be obtained from a subject, such as, for example, a subject or patient undergoing treatment for cancer. In some embodiments, a tissue biopsied from a subject may be used to prepare one or more 3D cell cultures of the present invention, optionally with cells obtained from a minced tissue.

Cells (e.g., live cells) may be incorporated into a composition and/or hydrogel of the present invention in any suitable form, including as unencapsulated cells, or as cells previously aggregated as spheroids, or pre-formed organoids. Animal tissue cells aggregated or contained in cell spheroids can be produced in accordance with known techniques, or in some cases are commercially available (see, e.g., Insphero AG, 3D Hepg2 Liver Microtissue Spheroids (2012); Inspherio AG, 3D InSight™ Human Liver Microtissues, (2012)).

“3D cell culture” or “three-dimensional tissue construct” as used herein refer to a plurality of live cells, optionally in a carrier media, that are arranged in a three-dimensional or multi-layered configuration (as opposed to a monolayer). An “organoid” as used herein refers to a composition of live cells, typically in a carrier media, arranged in a three-dimensional or multi-layered configuration (as opposed to a monolayer) and is a type of a 3D cell culture.

Suitable carrier media for a 3D cell culture include hydrogels (e.g., cross-linked hydrogels) as described herein. In some embodiments, a 3D cell culture is formed upon cross-linking (e.g., after UV initiated cross-linking) of the carrier media (e.g., hydrogel). Additional example hydrogels include, but are not limited to, those described in PCT/US2015/055699, PCT/US2016/054607, and PCT/US2017/058531, the contents of each of which are incorporated herein by reference in their entirety. A 3D cell culture may comprise one or more (e.g., 1, 2, 3, 4, or more) differentiated cell type(s) depending upon the particular tissue and/or organ being modeled or emulated. Some 3D cell cultures may comprise diseased cells and/or cancer cells. When the 3D cell culture comprises diseased cells and/or cancer cells, they may include tissue cells and/or may include a tissue mimic without cells, such as an extracellular matrix (or proteins and/or polymers derived therefrom), hyaluronic acid, gelatin, collagen, alginate, etc., including combinations thereof. Thus, in some embodiments, cells are mixed together with an extracellular matrix, or cross-linked matrix, to form a 3D cell culture.

In some embodiments, a 3D cell culture of the present invention comprises cells that are human-derived cells, and, in some embodiments, the cells consist of human-derived cells. A 3D cell culture of the present invention may express and/or produce one or more biomarkers (e.g., 1, 2, 3, 4, or more) that are the same as a biomarker produced by the cells in vivo. For example, liver cells in vivo produce albumin and a 3D cell culture of the present invention comprising liver cells may express albumin. In some embodiments, a 3D cell culture may express a biomarker in the same amount or in an amount that is ±20%, ±10%, or ±5% of the average amount produced and/or expressed by corresponding cells in vivo. Example biomarkers include, but are not limited to, albumin, urea, glutathione S-transferase (GST) (e.g., α-GST), chemokines (e.g., IL-8, IL-1β, etc.), prostacyclin, SB100B, neuron-specific enolase (NSE), myelin basic protein (MBP), hormones (e.g., testosterone, estradiol, progesterone, insulin, glucagon, etc.), inhibin A/B, lactate dehydrogenase (LDH), and/or tumor necrosis factor (TNF). The cells may be differentiated or undifferentiated cells, but are, in some embodiments, tissue cells (e.g., liver cells such as hepatocytes, pancreatic cells, cardiac muscle cells, skeletal muscle cells, etc.).

In some embodiments, a 3D cell culture of the present invention is not prepared from and/or does not comprise cells from an immortalized cell line. A 3D cell culture of the present invention may comprise and/or be prepared using high functioning cells, such as, but not limited to, primary cells and/or stem cells, e.g., embryonic stem cells, induced pluripotent stems and/or differentiated iPS-derived cells.

In some embodiments, one or more populations of cells may be labeled with a detectable compound. In some embodiments, the one or more populations of cells may be used to form a 3D cell culture as described herein. One or more different populations of cells in a 3D cell culture of the present invention may be present in substantially the same (e.g., within about ±20%) amount as the amount of cells in that population in a tissue and/or tumor in vivo. In some embodiments, when cells have been obtained from a tissue sample from a subject, sorted and/or labeled, the different populations of cells are combined in substantially the amount as the amount present in the tissue sample.

In some embodiments, an organoid is about 100, 200, or 300 μm to about 350, 400, 500, 600, or 700 μm in diameter in at least one dimension, such as, for example, about 100, 150, 200, 250, 300, 350, 400, 450, 500, 550, 600, 650, or 700 μm in at least one dimension. In some embodiments, an organoid is about 1 μL to about 20 μL in volume such as, for example, about 1, 2, 3, 4, 5, 6, 7, 8, 9, 10, 11, 12, 13, 14, 15, 16, 17, 18, 19, or 20 μL in volume. The organoid may comprise about 1,500, 2,000, or 5,000 to about 10,000, 25,000, or 50,000 cells in total or about 1,000, 5,000, 10,000, or 50,000 to about 75,000, 100,000, 150,000, 250,000, 500,000, 750,000, 1,000,000, 50,000,000, or 100,000,000 cells in total. In some embodiments, an organoid of the present invention may comprise about 1, 2, or 5 million to about 10, 50, or 100 million cells per mL. In some embodiments, an organoid of the present invention may comprise about 10 million cells per mL. An organoid of the present invention may be in any suitable shape, such as, e.g., any three-dimensional shape and/or multi-layered shape. In some embodiments, an organoid of the present invention is in the form of a spheroid. In some embodiments, an organoid of the present invention may be self-organized in a composition of the present invention (e.g., a cross-linked hydrogel).

“Growth media”, “liquid medium”, and “cell culture media”, along with grammatical variants thereof, are used interchangeably herein and may be any natural or artificial growth media (typically an aqueous liquid) that sustains the cells used in carrying out the present invention. Examples include, but are not limited to, an essential media or minimal essential media (MEM), or variations thereof such as Eagle's minimal essential medium (EMEM) and Dulbecco's modified Eagle medium (DMEM), as well as blood, blood serum, blood plasma, lymph fluid, etc., including synthetic mimics thereof. In some embodiments, the growth media includes a pH color indicator (e.g., phenol red).

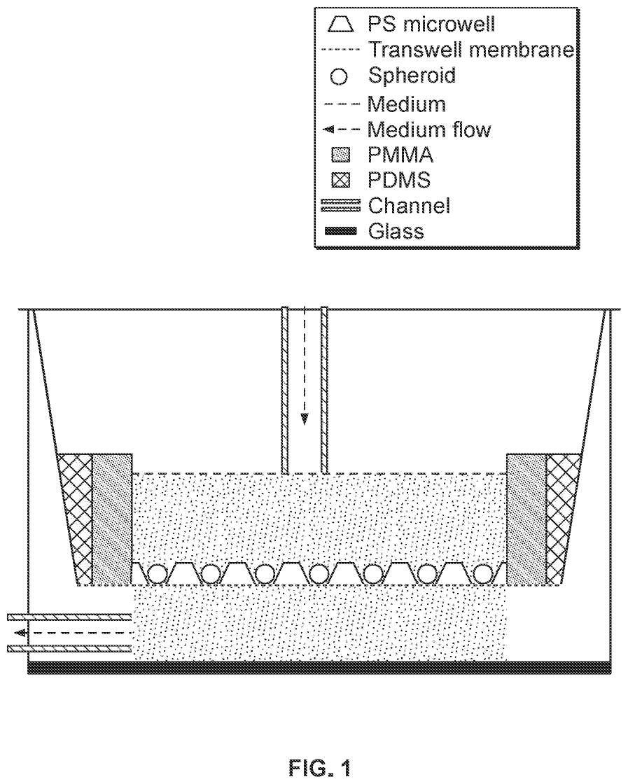

FIG. 1 is a general schematic of a microwell perfusion plate according to some embodiments. As described in more detail below, the plate may be provided in at least three configurations—static, (dynamic) perfusion with integrated cell culture chamber and fluidics, and (dynamic) perfusion with removable cell culture chamber (insert) and fluidics. Common to all three configurations is the use of a microwell through-pore membrane bonded onto a porous (polycarbonate) membrane.

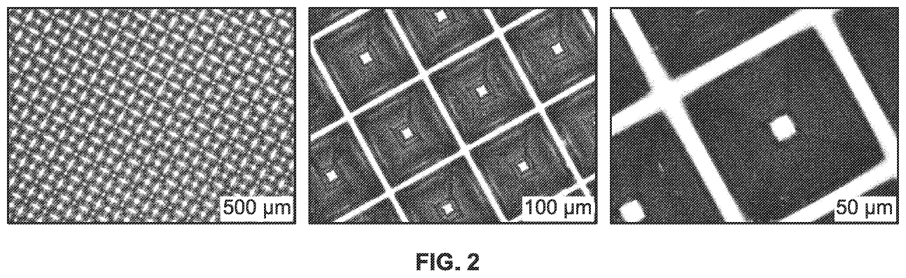

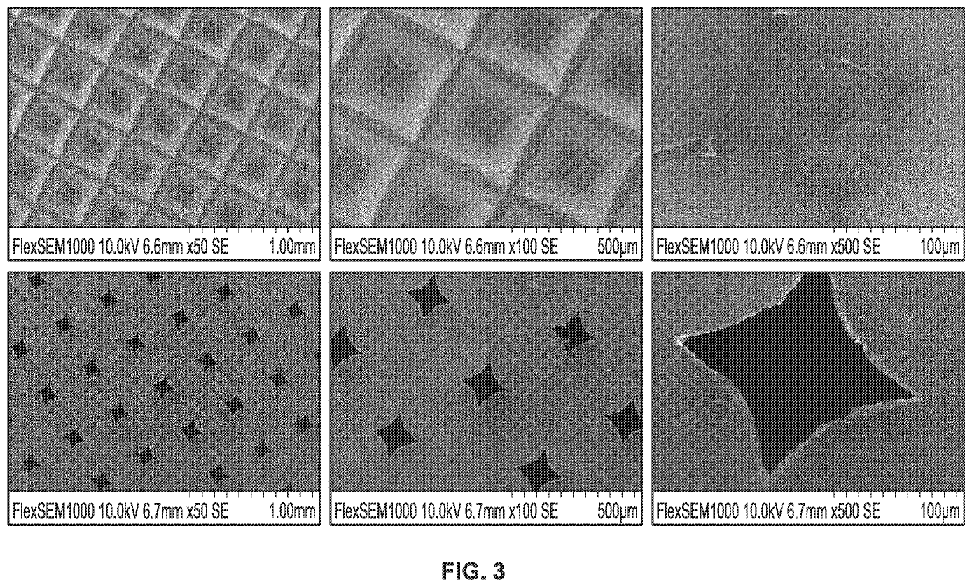

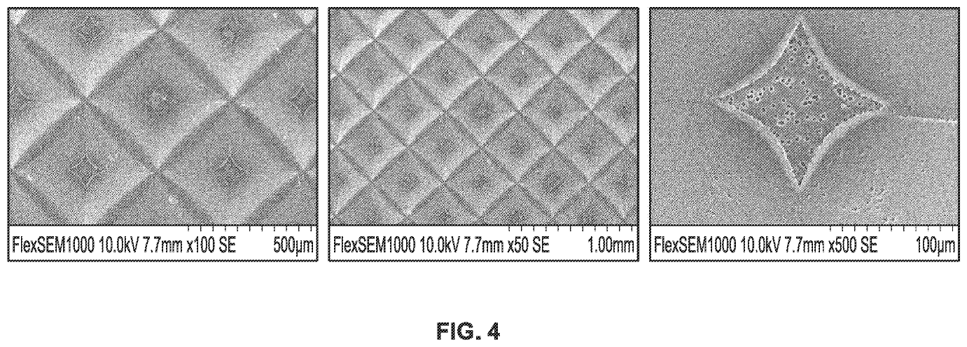

FIGS. 2 and 3 include digital images of the microwell through-pore membrane and FIG. 4 includes digital images of the microwell through-pore membrane bonded onto a porous polycarbonate membrane.

The microwell through-pore membrane has a top side and bottom side with openings or pores on both sides. The microwell membrane may have a thickness of about 200 μm. The top opening may be about 400 μm (side measurement). The bottom opening may be between about 40 μm and 150 μm (side measurement). In some embodiments, the bottom opening is about 100 μm.

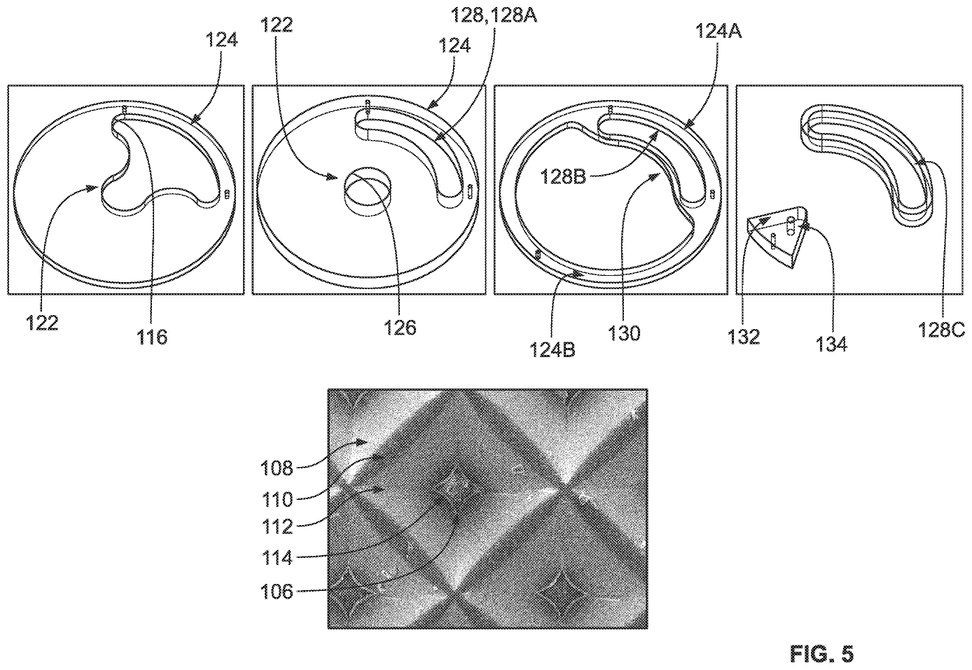

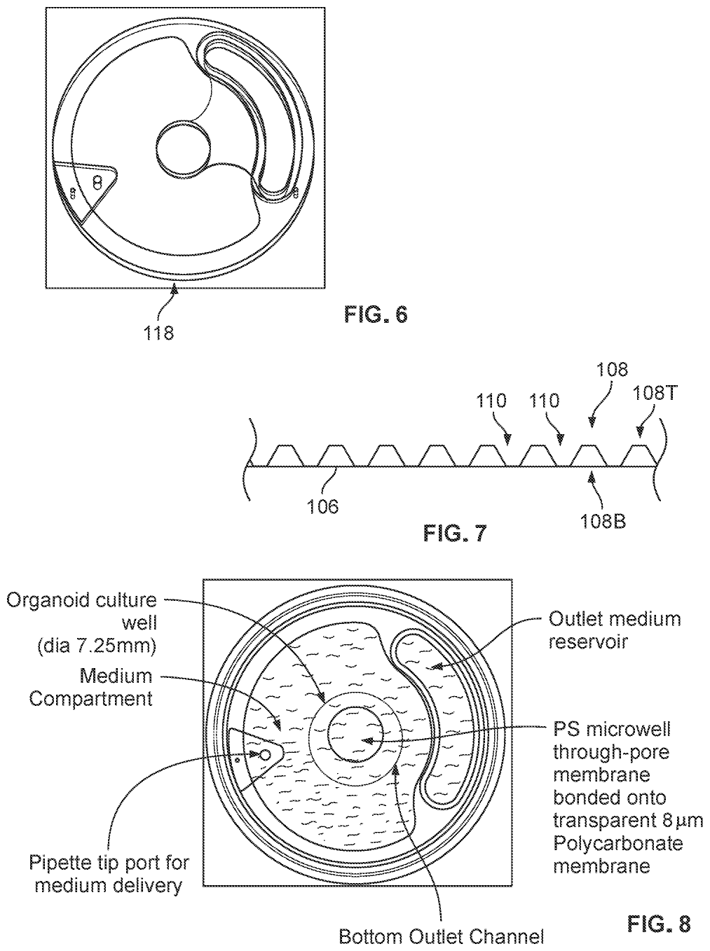

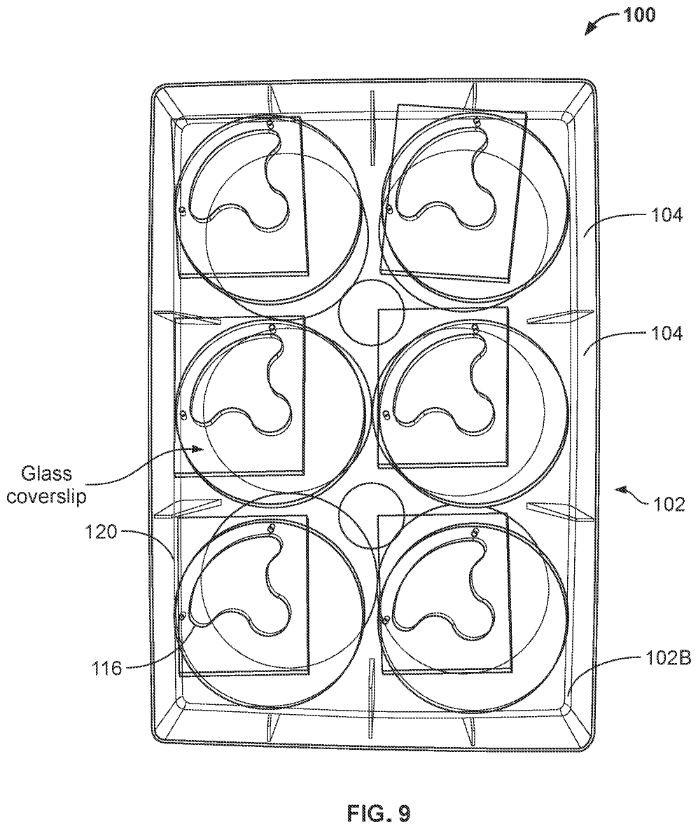

FIGS. 5-9 illustrate the static microwell perfusion plate. Referring to FIG. 9, a system 100 includes a plate 102 and at least one compartment or well 104 on the plate 102. As illustrated, there may be a plurality of wells 104 on the plate 102. For example, there may be 2, 4, 6, 8, 10, 12, or more wells on the plate.

Each well 104 includes the porous membrane 106 and the microwell through-pore membrane 108 that are also shown in FIGS. 2-4.

Referring to FIGS. 5 and 7, the microwell membrane 108 includes a top 108T and a bottom 108B. The microwell membrane includes an array of a plurality of microwells 110. Each microwell 110 includes a top opening 112 at the top 108T and a bottom opening 114 at the bottom 108B.

The microwell membrane 108 is above and on the porous membrane 106.

The well 104 includes a bottom outlet channel 116 and a body 118 with the porous membrane 106 and the microwell through-pore membrane 108 positioned between the bottom outlet channel 116 and the body 118. Referring to FIG. 9, the bottom outlet channel 116 may be cut in the plate 102. A glass coverslip 120 may be bonded to a bottom side 102B of the plate 102. This configuration may provide enhanced imaging (e.g., as compared to having an additional layer with the bottom outlet channel defined in the additional layer).

The plate 102 may be transparent or substantially transparent. A suitable material is polystyrene.

The bottom outlet channel 116 is below the porous membrane 106. The bottom outlet channel 116 may extend between a central portion 122 of the well 104 (or the body 118) and an outer peripheral portion 124 of the well 104 (or the body 118). As illustrated, the bottom outlet channel 118 may widen from the central portion 122 to the outer peripheral portion 124. This configuration increases the volume of medium in the static plate design and may reduce the need to frequently change the medium.

A cell culture chamber or well 126 is defined in the central portion 122 of the body 118. The cell culture well 126 is above the microwell membrane 108. In some other embodiments, there may be a plurality of cell culture wells (smaller in diameter than represented in this design, in order to accommodate the plurality), with each cell culture well having a plurality of microwells (e.g., above a microwell membrane as described herein).

An outlet medium reservoir 128 is defined in the outer peripheral portion 124 of the body 118. The outlet medium reservoir 128 is in fluid communication with and positioned above the bottom outlet channel 116 at the outer peripheral portion 124 of the well 104. The outlet medium reservoir 128 may be arcuate and extend along the outer peripheral portion 124 of the body 118. This configuration also increases the volume of medium in the static plate design.

An inlet medium compartment 130 is defined in the body 118. The inlet medium compartment 130 is in fluid communication with and positioned above the cell culture well 126.

The outlet medium reservoir 128 is at a first side 124A of the outer peripheral portion 124 of the body 118. The inlet medium compartment 130 extends between the outlet medium reservoir and a second, opposite side 124B of the outer peripheral portion 124 of the body 118. The relatively large size of the inlet medium compartment 130 further increases the volume of medium in the static plate design.

An inlet port member 132 including an inlet port 134 is at the second side 124B of the outer peripheral portion 124 of the body 118. The inlet port 134 is in fluid communication with and positioned above the inlet medium compartment 130. The inlet port 134 is configured to receive a pipette tip to deliver liquid medium to the inlet medium compartment 130.

The inlet port member 132 is off to the side because medium delivered by a pipette may dislodge or otherwise disturb the cells in the microwell membrane 108 if the inlet port was positioned directly over the cell culture well 126.

The outlet medium reservoir 128 may include a lower portion 128A, an intermediate portion 128B, and an upper portion 128C.

The body 118 is shown as including a number of layers in the embodiment shown in FIG. 5. In some other embodiments, the body 118 including the cell culture well 126, the outlet medium reservoir 128, the inlet medium compartment 130, and/or the inlet port member 132 may be monolithic.

As used herein, the “inlet passageway” or “inlet fluid passageway” may be defined by the cell culture well 126, the inlet medium compartment 130, and/or the inlet port 134. As used herein, the “outlet passageway” or “outlet fluid passageway” may include the outlet medium reservoir 128.

The body 118 is preferably formed of a transparent or substantially transparent material for enhanced imaging. A suitable material is PMMA.

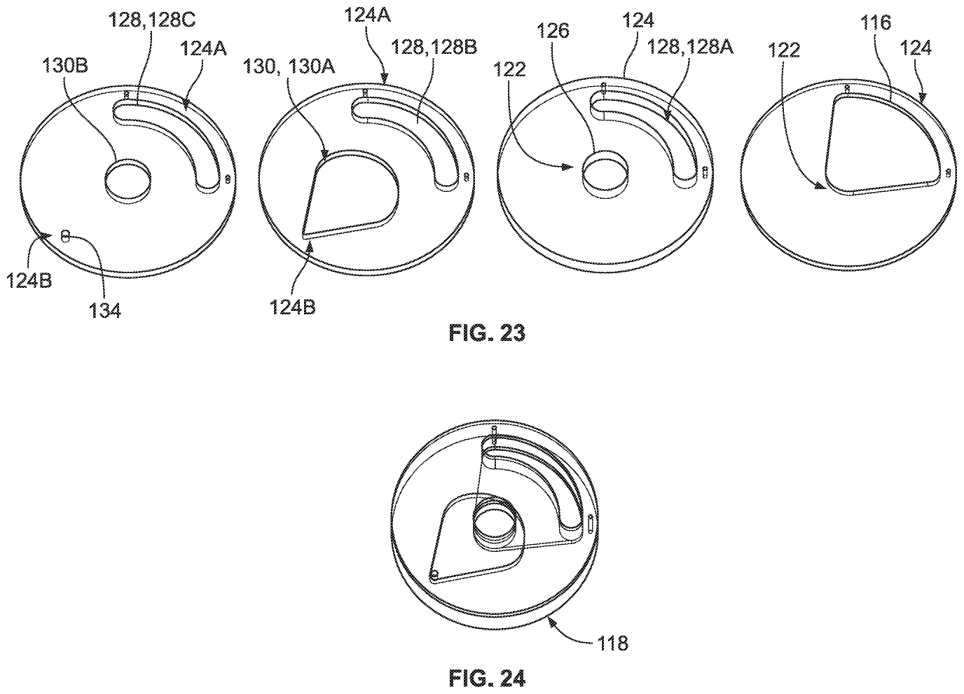

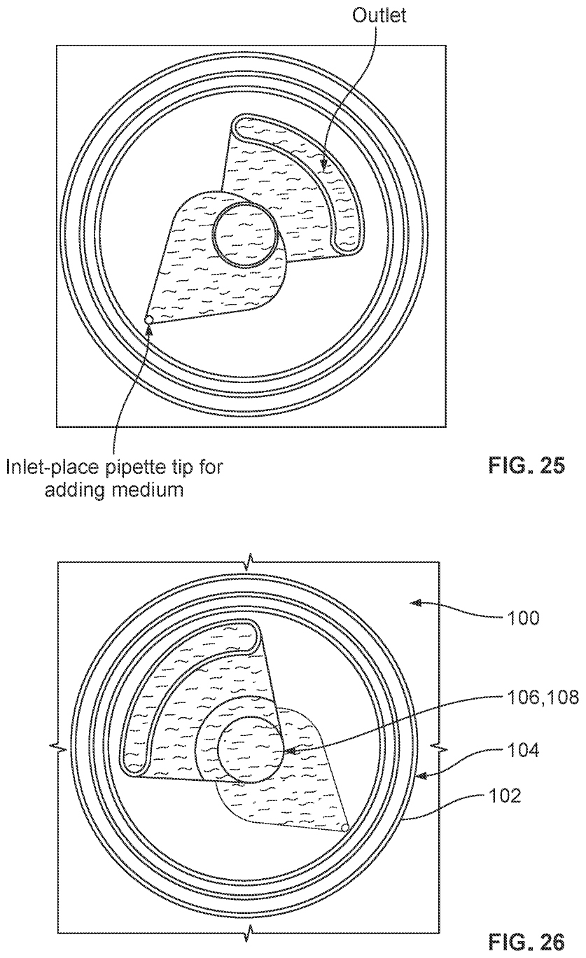

FIGS. 23-26 illustrate the static microwell perfusion plate according to another embodiment. Referring to FIGS. 9 and 26, the plate design can be used with the system 100 that includes a plate 102 and at least one (or a plurality of) compartment or well 104 on the plate 102. As illustrated, there may be a plurality of wells 104 on the plate 102. For example, there may be 2, 4, 6, 8, 10, 12, or more wells on the plate.

Each well 104 includes the porous membrane 106 and the microwell through-pore membrane 108 that are also shown in FIGS. 2-4.

Referring to FIGS. 5 and 7, the microwell membrane 108 includes a top 108T and a bottom 108B. The microwell membrane includes an array of a plurality of microwells 110. Each microwell 110 includes a top opening 112 at the top 108T and a bottom opening 114 at the bottom 108B.

The microwell membrane 108 is above and on the porous membrane 106.

The well 104 includes a bottom outlet channel 116 and a body 118 with the porous membrane 106 and the microwell through-pore membrane 108 positioned between the bottom outlet channel 116 and the body 118. Similar to FIG. 9, the bottom outlet channel 116 may be cut in the plate 102. A glass coverslip 120 may be bonded to a bottom side 102B of the plate 102. This configuration may provide enhanced imaging (e.g., as compared to having an additional layer with the bottom outlet channel defined in the additional layer).

The plate 102 may be transparent or substantially transparent. A suitable material is polystyrene.

The bottom outlet channel 116 is below the porous membrane 106. The bottom outlet channel 116 may extend between a central portion 122 of the well 104 (or the body 118) and an outer peripheral portion 124 of the well 104 (or the body 118). As illustrated, the bottom outlet channel 118 may widen from the central portion 122 to the outer peripheral portion 124. This configuration increases the volume of medium in the static plate design and may reduce the need to frequently change the medium. This design also provides improved fluid communication and helps to prevent formation of air pockets.

A cell culture chamber or well 126 is defined in the central portion 122 of the body 118. The cell culture well 126 is above the microwell membrane 108. In some other embodiments, there may be a plurality of cell culture wells (smaller in diameter than represented in this design, in order to accommodate the plurality), with each cell culture well having a plurality of microwells (e.g., above a microwell membrane as described herein).

An outlet medium reservoir 128 is defined in the outer peripheral portion 124 of the body 118. The outlet medium reservoir 128 is in fluid communication with and positioned above the bottom outlet channel 116 at the outer peripheral portion 124 of the well 104. The outlet medium reservoir 128 may be arcuate and extend along the outer peripheral portion 124 of the body 118. This configuration also increases the volume of medium in the static plate design.

An inlet medium compartment 130 is defined in the body 118. The inlet medium compartment 130 is in fluid communication with and positioned above the cell culture well 126.

The outlet medium reservoir 128 is at a first side 124A of the outer peripheral portion 124 of the body 118. The inlet medium compartment 130 extends between the central portion 122 of the body 118 and a second, opposite side 124B of the outer peripheral portion 124 of the body 118. The inlet medium compartment 130 may narrow from the central portion 122 of the body 188 to the second side 124B of the outer peripheral portion 124 of the body 118. The relatively large size of the inlet medium compartment 130 further increases the volume of medium in the static plate design.

An inlet port 134 is at the second side 124B of the outer peripheral portion 124 of the body 118. The inlet port 134 is in fluid communication with and positioned above the inlet medium compartment 130. The inlet port 134 is configured to receive a pipette tip to deliver liquid medium to the inlet medium compartment 130.

The inlet port 134 is off to the side because medium delivered by a pipette may dislodge or otherwise disturb the cells in the microwell membrane 108 if the inlet port was positioned directly over the cell culture well 126.

The outlet medium reservoir 128 may include a lower portion 128A, an intermediate portion 128B, and an upper portion 128C.

The inlet medium compartment 130 may be a first inlet medium compartment 130A. A second inlet medium compartment 130B may be in the central portion 122 of the body 118. The second inlet medium compartment 130B is in fluid communication with and positioned above the first inlet medium compartment 130A. The use of first and second inlet medium compartments 130A, 130B further increases the volume of medium in the static plate design. This design including the top layer also allows for easier fluid flow and communication from the inlet port 134, through the inlet medium compartment 130, and into cell culture well 126.

The body 118 is shown as including a number of layers in the embodiment shown in FIG. 24. In some other embodiments, the body 118 including the cell culture well 126, the outlet medium reservoir 128, the inlet medium compartment 130, and/or the inlet port 134 may be monolithic.

As used herein, the “inlet passageway” or “inlet fluid passageway” may be defined by the cell culture well 126, the inlet medium compartment 130, and/or the inlet port 134. As used herein, the “outlet passageway” or “outlet fluid passageway” may include the outlet medium reservoir 128.

The body 118 is preferably formed of a transparent or substantially transparent material for enhanced imaging. A suitable material is PMMA. In some embodiments, the body 118 can be 3D printed in two parts (top and bottom) rather than multiple layers that are laser cut. However, it may be translucent rather than transparent given the present unavailability of fully transparent ink or resin.

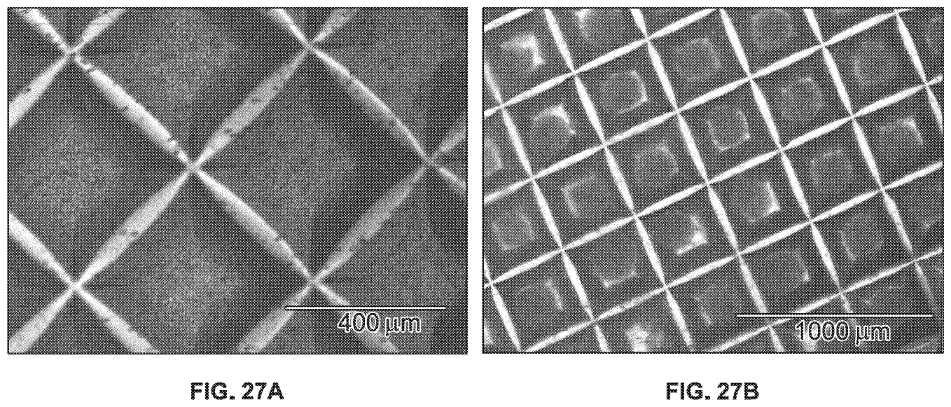

FIG. 27A is a digital image illustrating human embryonic stem cells (hESC) seeded at a density of 2000 cells/microwell showing uniform distribution in the static microwell perfusion plate of FIGS. 23-26. FIG. 27B is a digital image illustrating the formation of homogenously-sized spheroids after 48 hours.

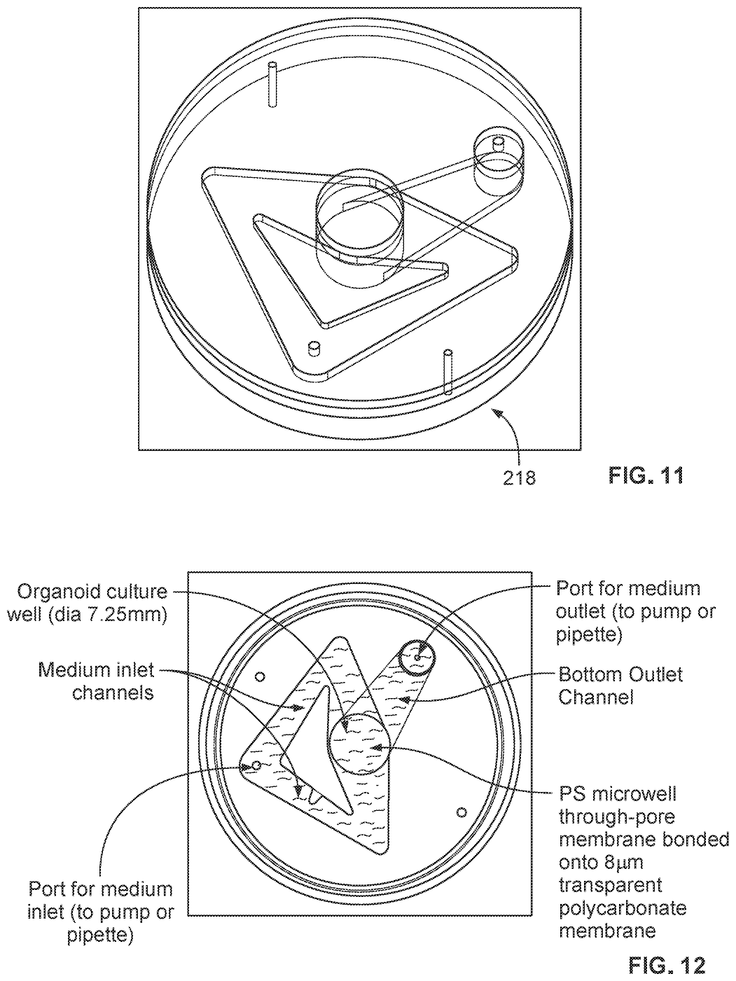

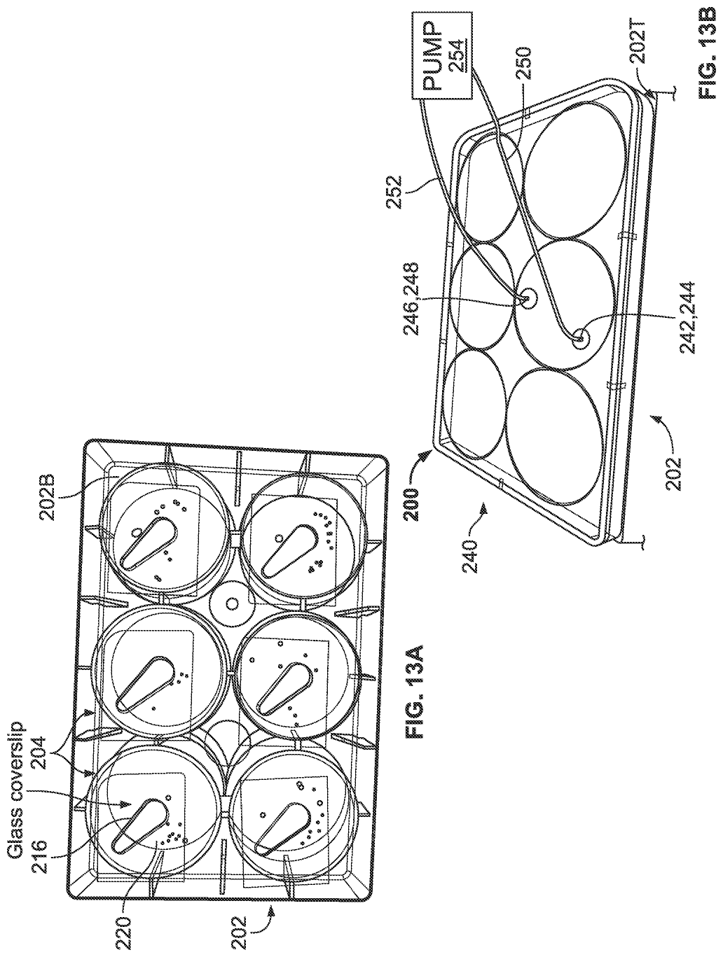

FIGS. 7 and 10-13 illustrate the dynamic perfusion plate with integrated cell culture chamber and fluidics. Referring to FIG. 13A, a system 200 includes a plate 202 and at least one compartment or well 204 on the plate 202. As illustrated, there may be a plurality of wells 204 on the plate 202. For example, there may be 2, 4, 6, 8, 10, 12, or more wells on the plate.

Each well 204 includes the porous membrane 106 and the microwell through-pore membrane 108 that are also shown in FIGS. 2-4.

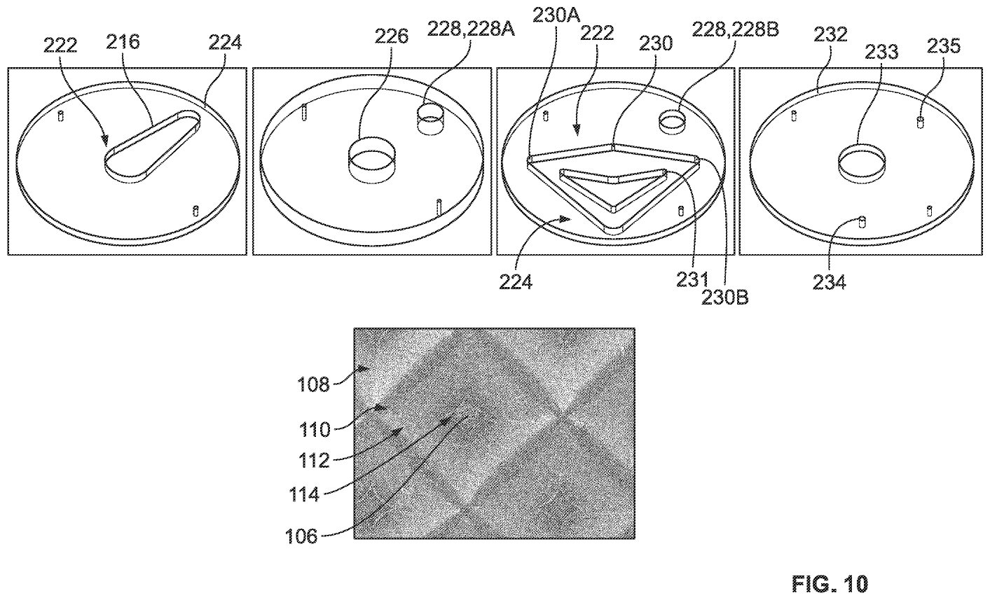

Referring to FIGS. 7 and 10, the microwell membrane 108 includes a top 108T and a bottom 108B. The microwell membrane includes an array of a plurality of microwells 110. Each microwell 110 includes a top opening 112 at the top 108T and a bottom opening 114 at the bottom 108B.

The microwell membrane 108 is above and on the porous membrane 106.

The well 204 includes a bottom outlet channel 216 and a body 218 with the porous membrane 106 and the microwell through-pore membrane 108 positioned between the bottom outlet channel 216 and the body 218. Referring to FIG. 13, the bottom outlet channel 216 may be cut in the plate 202. A glass coverslip 220 may be bonded to a bottom side 202B of the plate 202. This configuration may provide enhanced imaging (e.g., as compared to having an additional layer with the bottom outlet channel defined in the additional layer).

The plate 202 may be transparent or substantially transparent. A suitable material is polystyrene.

The bottom outlet channel 216 is below the porous membrane 106. The bottom outlet channel 216 may extend between a central portion 222 of the well 204 (or the body 218) and an outer peripheral portion 224 of the well 204 (or the body 218). As illustrated, the bottom outlet channel 218 may narrow from the central portion 222 to the outer peripheral portion 224. In some other embodiments, the bottom outlet channel may have a constant width or substantially constant width between the central portion 222 and the outer peripheral portion 224. These configurations reduce the residence volume of medium in the dynamic plate design (e.g., compared to the static plate design described above) and reduces “dead space” where the medium does not flow or flows inefficiently.

A cell culture chamber or well 226 is defined in the central portion 222 of the body 218. The cell culture well 226 is above the microwell membrane 108.

An outlet medium reservoir 228 is defined in the outer peripheral portion 224 of the body 218. The outlet medium reservoir 228 is in fluid communication with and positioned above the bottom outlet channel 216 at the outer peripheral portion 224 of the well 204. The outlet medium reservoir 228 may be circular. As compared to the outlet medium reservoir 128 described above, the outlet medium reservoir 228 may be smaller to reduce the residence volume of medium in the dynamic plate design to thereby reduce “dead space” where the medium does not flow or flows inefficiently.

An inlet medium compartment 230 is defined in the body 218. The inlet medium compartment 230 is in fluid communication with and positioned above the cell culture well 226. The inlet medium compartment 230 may diverge into first and second inlet fluid pathways 230A, 230B at the outer peripheral portion 224 of the body 218 and the first and second inlet fluid pathways 230A, 230B may converge at the central portion 222 of the body 218. This configuration allows medium to enter the cell culture well 226 from two sides and may provide improved direct flow over all the cells in the microwell membrane 108. A member such as a triangle member 231 may be used to bifurcate the inlet medium compartment 230. In some other embodiments, the inlet medium passageway 230 may have a configuration and shape similar to that of the inlet medium passageway 130 described above.

An inlet and outlet port member 232 includes an inlet port 234 and an outlet port 235. The inlet port 234 is in fluid communication with and positioned above the inlet medium compartment 230. The outlet port 235 is in fluid communication with and positioned above the outlet medium reservoir 228.

An opening 233 may be defined in the top of the body 228. The opening 233 may be positioned above and aligned with the cell culture well 226 to, for example, provide access to the cells in the microwell membrane 108.

The outlet medium reservoir 128 may include a lower portion 228A and an upper portion 228B.

The body 218 is shown as including a number of layers in the embodiment shown in FIG. 10. In some other embodiments, the body 218 including the cell culture well 226, the outlet medium reservoir 228, the inlet medium compartment 230, and/or the inlet and outlet port member 232 may be monolithic.

The body 218 is preferably formed of a transparent or substantially transparent material for enhanced imaging. A suitable material is PMMA. In some embodiments, the body 218 can be 3D printed in two parts (top and bottom) rather than multiple layers that are laser cut. However, it may be translucent rather than transparent given the present unavailability of fully transparent ink or resin.

Referring to FIG. 13B, the system 200 includes a lid 240 that is configured to be selectively installed over a top 202T of the plate 202. An inlet port 242 and an outlet port 246 are defined in the lid 240 for each well 204. An inlet coupler 244 (e.g., a metal coupler) is in the inlet port 242 and an outlet coupler 248 (e.g., a metal coupler) is in the outlet port 246. The inlet port 242 and the outlet port 246 are in fluid communication with at least one pump 254. For example, an inlet tube 250 (e.g., a silicon tube) may be connected to the inlet coupler 244 and the pump 254 and an outlet tube 252 (e.g., a silicon tube) may be connected to the outlet coupler 248 and the pump 254.

Referring to FIGS. 10 and 13B, with the lid 240 placed in the installed position on the plate 202, the inlet port 234 of the body 218 (or the plate 202) is aligned with the inlet port 242 of the lid 240 and the outlet port 235 of the body 218 (or the plate 202) is aligned with the outlet port 246 of the lid 240. In this way, the pump 254 can be operated (e.g., continuously) to deliver and retrieve medium from the system.

In some embodiments, the couplers 244, 248 extend downwardly from the lid 240 and are received in the inlet port 234 and the outlet port 235, respectively, when the lid 240 is placed in the installed position with the lid 240 on the plate 202.

As used herein, the “inlet passageway” or “inlet fluid passageway” may be defined by the cell culture well 226, the inlet medium compartment 230, the inlet port 234 of the body 218, the inlet port 242 of the lid 240, and/or the inlet coupler 244. As used herein, the “outlet passageway” or “outlet fluid passageway” may include the outlet medium reservoir 228, the outlet port 235 of the body 218, the outlet port 246 of the lid 240, and/or the outlet coupler 248.

Because this is an open system, a user can remove the lid 240, optionally install a standard lid (without the couplers and tubing), and perform imaging. This may be beneficial if imaging without perfusion is desired.

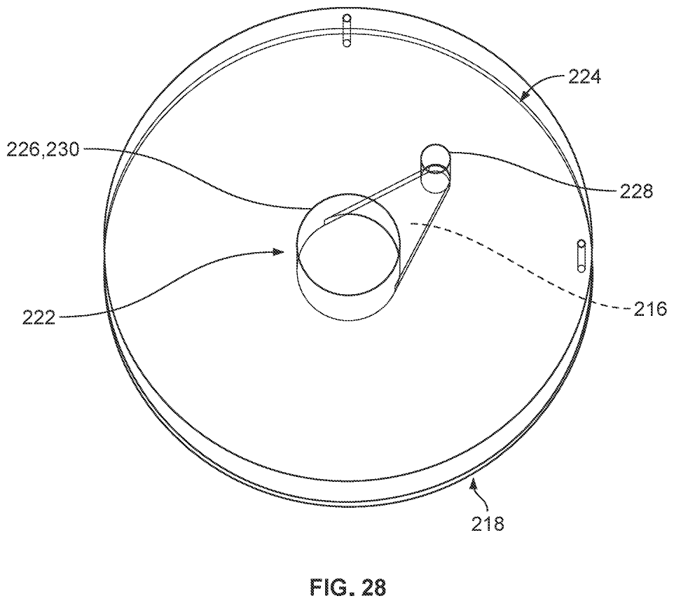

FIG. 28 illustrates an alternative embodiment of the body 218 that can be used with the dynamic perfusion plate system of FIGS. 10-13. The bottom outlet channel 216 and the outlet medium reservoir 228 may be similar to those in the embodiment of FIGS. 10-13, although the outlet medium reservoir 228 may be closer to the central portion 222 of the body 218. For example, the outlet medium reservoir 228 may be between the central portion 222 of the body 218 and the outer peripheral portion 224 of the body 218. In addition, the inlet medium compartment 230 is positioned above the cell culture well 226 rather than partially off to the side. This design further reduces the medium residence volume. A top layer, such as the one shown in FIG. 10 including the inlet port 234 and the outlet port 235 may be included as part of the body 218. However, the inlet port 234 may be positioned above the cell culture well 226 and/or the inlet medium compartment 230 and the opening 233 may be omitted. As described above with regard to FIGS. 10-13, the body 218 may be part of a plate system that may be connected to a pump (e.g., a peristaltic pump) via tubing (e.g., silicone tubing) that is inserted (and sealed) through holes in the plate lid to feed/remove culture medium directly to the culture well and outlet, respectively. The feed rate of the pump can be adjusted to maintain a constant media height, thus conferring a constant flow rate.

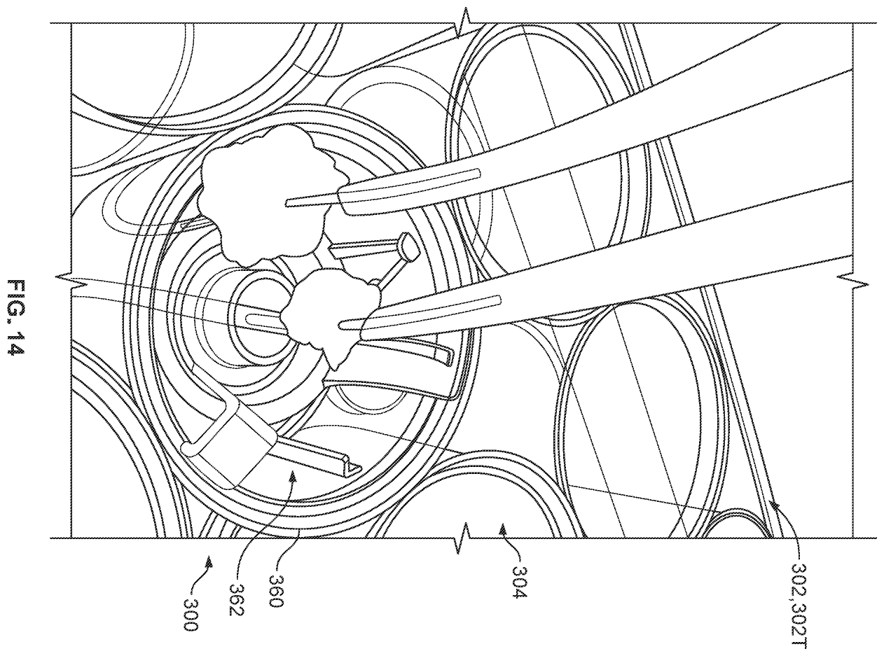

FIGS. 14-18 illustrate the dynamic perfusion system with removable cell culture chamber (insert) and fluidics. The system 300 may be a modified Transwell system available from Corning. In the Transwell system, the plate 302 includes a plurality of compartments or wells 304 with each well 304 including a container 360 in which an inert 362 is received.

Referring to FIGS. 15-17, the insert 362 is modified to include the porous membrane 106 and the microwell through-pore membrane 108 that are also shown in FIGS. 2-4 and 7. The microwell membrane 108 is above and on the porous membrane 106.

A body 318 is above and on the microwell membrane 108 and/or the insert 362. The body includes a sidewall 319 that surrounds the porous membrane 106 and the microwell membrane 108. The sidewall 319 may be in the shape of a cylinder and may be formed of any suitable material; an example material is PMMA.

In some embodiments, the body 318 includes a second sidewall 317 surrounding the (first) sidewall 319. The second sidewall 317 may be useful to help prevent spilling or ingress of medium into the outlet reservoir. A suitable material for the second sidewall 317 is PDMS.

The body 318 defines a cell culture chamber or well 326 above the porous membrane 106 and the microwell membrane 108. The container 360 defines an outlet medium reservoir 328 below the porous membrane 106 and the microwell membrane 108.

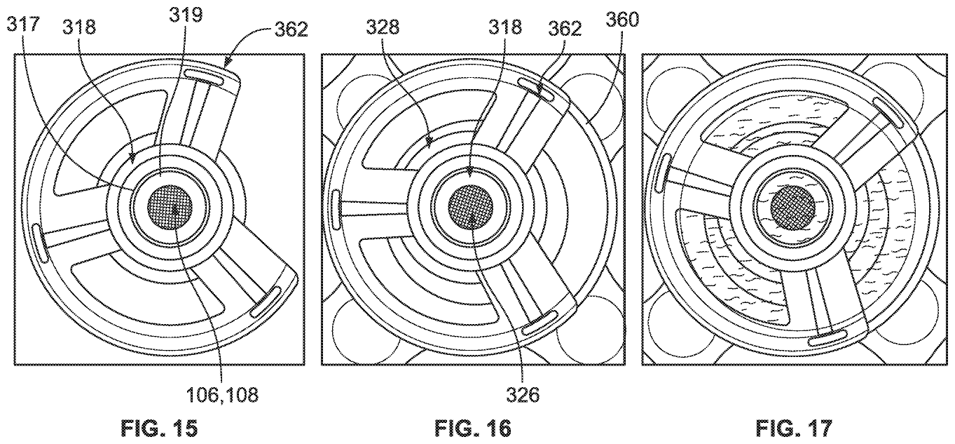

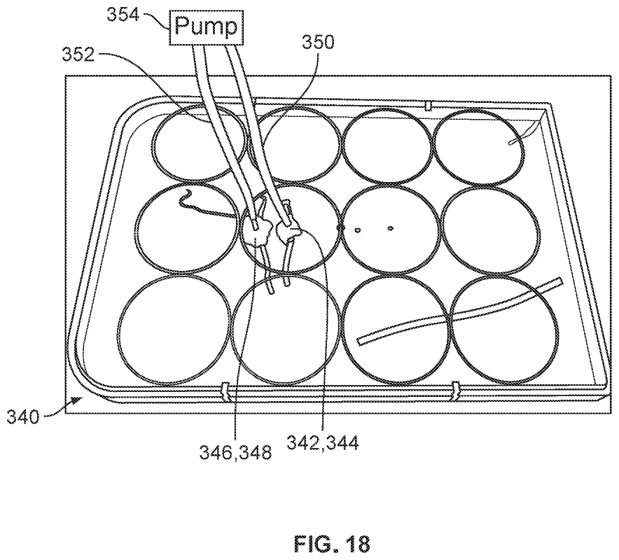

Referring to FIG. 18, the system 300 includes a lid 340 that is configured to be selectively installed over a top 302T of the plate 302 (FIG. 14). An inlet port 342 and an outlet port 346 are defined in the lid 340 for each well 304. An inlet coupler 344 (e.g., a metal coupler) is in the inlet port 342 and an outlet coupler 348 (e.g., a metal coupler) is in the outlet port 346. The inlet port 342 and the outlet port 346 are in fluid communication with at least one pump 354. For example, an inlet tube 350 (e.g., a silicon tube) may be connected to the inlet coupler 344 and the pump 354 and an outlet tube 352 (e.g., a silicon tube) may be connected to the outlet coupler 348 and the pump 354.

Referring to FIGS. 14 and 18, with the lid 340 placed in the installed position on the plate 302, the inlet port 342 of the lid 340 is above and aligned with the cell culture well 326. In some embodiments, the inlet coupler 344 extends downwardly from the lid 340 and into or toward the cell culture well 326. Also with the lid 340 placed in the installed position on the plate 302, the outlet port 246 of the lid 240 is above and aligned with the outlet medium reservoir 328. In some embodiments, the outlet coupler 348 extends downwardly from the lid 340 and into or toward the outlet medium reservoir 328. With these configurations, the pump 354 can be operated (e.g., continuously) to deliver and retrieve medium from the system.

As used herein, the “inlet passageway” or “inlet fluid passageway” may be defined by the cell culture well 326, the inlet port 342 of the lid 340, and/or the inlet coupler 344. As used herein, the “outlet passageway” or “outlet fluid passageway” may include the outlet medium reservoir 328, the outlet port 346 of the lid 340, and/or the outlet coupler 348.

Because this is an open system, a user can remove the lid 340, optionally install a standard lid (without the couplers and tubing), and perform imaging from the top. This may be beneficial if imaging without perfusion is desired.

The system 300 allows the insert 352 and thus the microwell membrane 108 to be removed for imaging or other processing.

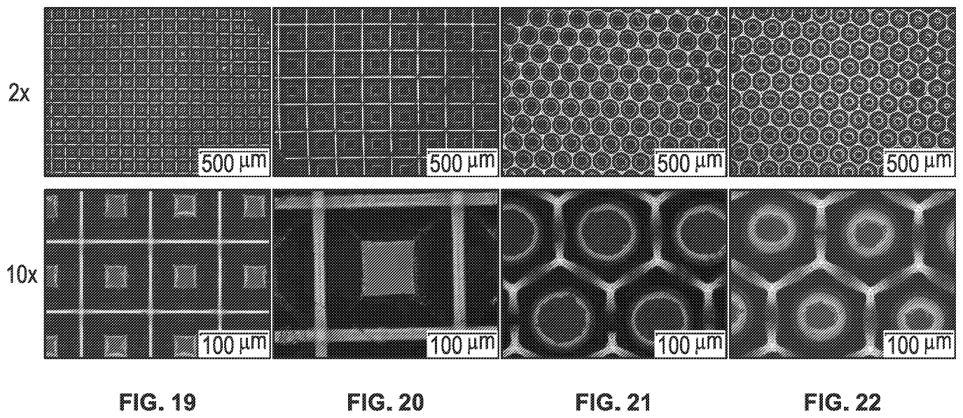

The microwell membrane 108 shown in, for example, FIGS. 2-4, includes pyramidal microwells with generally square openings or pores. Some alternative designs are illustrated in FIGS. 19-22. In FIG. 19, the microwell membrane includes small square openings or pores and sloped, trapezoidal sidewalls. In FIG. 20, the microwell membrane includes large square openings or pores and sloped, trapezoidal sidewalls. In FIG. 21, the microwell membrane includes large circular or round openings or pores and curved microwell sidewalls. The microwells are hemispherical. In FIG. 22, the microwell membrane includes small circular or round openings or pores and curved microwell sidewalls. The microwells are hemispherical.

It is believed that one or more of these designs may be desirable as the microwells more closely match the shape of spheroids.

Current commercial microwells (e.g., Aggrewell available from Stemcell Technologies) and microwell-based methods for producing microtissues are known, but can have disadvantages. Such methods may provide homogenous spheroid size, low cell loss, easy retrieval of spheroids, and scalability. However, such methods introduce a number of disadvantages. Well volume is optimized for monolayer culture. There is a high cell volume to surface ratio due to cells cultured as spheroids rather than monolayer. There is a high bioburden leading to frequent medium change. Spheroids tend to “pop out” during medium change. There is retention of cell debris in the microwells. They provide short term culture (spheroids have to be removed and cultured in another platform after formation). There is a lack of fluid flow (perfusion) leading to inadequate nutrient and oxygen exchange and waste removal. There are also inadequate mechanical forces due to the absence of fluid flow.

The microwell perfusion plates described herein combine the advantages of microfluidics, bioreactors, and microwell platforms. The microwell perfusion plates provide an open-well plate rather than a closed organ-on-chip. The plates take advantage of a standard multi-well plate footplate to use existing imaging setups. The plates enable easy cell seeding, media sampling, and recovery of spheroids for downstream processing (e.g., bioprinting). The plates provide a reduced bioburden due to perfusion. The plates allow for easier application/change of growth factors/medium during different stages of differentiation and maturation. The spheroids can be encapsulated/embedded in the plate in the form of a sheet. The plates provide direct flow of medium rather than tangential flow (e.g., associated with microfluidic chips which provides an inadequate exchange due to lack of convective flow that can create dead zones in microwells). All cell debris can be removed due to pore-through microwell that is bonded to the porous membrane. The plates allow for in-plate readouts for imaging based or dynamic (real time course) assays (e.g., GSIS assays). The plates may be fabricated with PS and/or PMMA to avoid absorption and/or leaching associated with PDMS (conventional material).

The microwell perfusion plates and systems described herein support the formation, dynamic testing and simultaneous live-cell imaging of microtissues, and may prevent their agglomeration, which could lead to diffusion limitations. The design enables direct flow of medium (through the microwells) rather than tangential, eliminating the possibility of inadequate medium exchange associated with traditional microwell-based microfluidic devices. The direct medium flow also provides mechanical cues and allows for removal of cell debris otherwise trapped in the microwells. The plates may be fabricated using rapid prototyping techniques, with non-absorbent and inert materials (polystyrene; PS & acrylic; PMMA). The platform has the footprint of a standard multiwell plate; therefore, it is compatible with standard imaging platforms for time-resolved assessment of cellular readouts, and rapid assessment of organoid functionality. The design being an open-well plate, rather than a closed organ-on-chip, enables manual access for cell seeding, sampling and recovery of microtissues for analysis (histology; electron microscopy; DNA, RNA and protein extraction) or other applications like 3D bioprinting and implantation.

The following non-limiting examples illustrate the use of the perfusion plate systems described herein.

### EXAMPLES

### Example 1

The limited availability of human organ donors renders islet allotransplantation unlikely to provide a cure for uncomplicated type 1 diabetes (T1D). Consequently, there is a significant interest in alternative sources of insulin producing cells (IPCs) and research efforts have focused intensively on generating functional β cells or endocrine cell clusters from stem cells. However, the major barrier in generating IPCs is the considerably low yield of the differentiation process and the inability to sustain mature β cells in culture. Recent studies have suggested that differentiation in 3D could result in improved efficiency and higher yield. Moreover, perfusion can also improve the long term viability and function of microtissues, by providing an adequate nutrient and oxygen supply and waste removal. The use of a culture platform that enables the formation of homogenously sized 3D microtissues and allows for continuous medium perfusion.

The 3D microwell-based perfusion plate supports the formation, dynamic testing and simultaneous live-cell imaging of islet-like clusters (ILCs).

The microwell platform would enable the formation and guided differentiation of homogenously sized ILCs from human embryonic stem cell (hESC)—derived pancreatic progenitor (PP) cells, while preventing their agglomeration, which could lead to diffusion limitations. The platform geometry enables direct flow of medium (through the microwells) rather than tangential (across; FIG. 1a, b), eliminating the possibility of inadequate medium exchange associated with traditional microwell-based devices. The plate was fabricated using rapid prototyping techniques, like laser machining and hot embossing, using non-absorbent and inert materials (polystyrene; PS & acrylic; PMMA). The platform is compatible with standard imaging platforms for time-resolved assessment of cellular readouts, and rapid assessment of islet functionality, such as dynamic GSIS. The design being an open-well plate, rather than a closed organ-on-chip, enables manual access for cell seeding, sampling and recovery of ILCs for analysis or other applications like 3D bioprinting and implantation.

At Stage 5 of differentiation, PP cells are transferred to non-adherent growth platforms, resulting in cluster formation. As there is no physical constraint on size and separation, they can grow over time or agglomerate, leading to diffusion limitations. Microwell arrays that physically restrict spheroid size can be used for achieving a defined and homogenous size. They can also be incorporated in microfluidic devices to sequester spheroids in individual microwells, provide control over fluid flow and test islets a dynamic manner. However, there are several disadvantages associated with conventional microfluidic devices. Medium flow across the face of microwell (tangential) can be inadequate in providing complete medium exchange, especially for deeper microwells. It has been shown that devices with islets trapped in cup shaped nozzles (open on both ends) stimulated intracellular flow, resulting in enhanced β-cell preservation. Moreover, permanent bonding of devices can make cell loading and recovery difficult. Fabrication requires cleanrooms for soft lithography, and the fabrication material (polydimethylsiloxane; PDMS) is unsuitable due to absorption of hydrophobic reagents and leaching of small molecules. Finally, such devices might be suited for testing but cannot be easily scaled up for biomanufacturing ILCs in physiomimetic conditions, discouraging their widespread adoption.

PP cells (Stage 4) are seeded in the microwell platform to enable the formation of homogenously sized spheroids. These can be further differentiated either in the presence of soluble human pancreatic ECM, embedded in ECM hydrogel, or a combination of both, and exposed to different flow rates. Without wishing to be bound by theory, we hypothesize that the combination of microenvironmental cues from the ECM and medium flow will result in a higher yield of insulin producing cells, compared to control conditions.

Pancreas decellularization and ECM preparation: Briefly, human pancreas from disease free organ donors is obtained from the local organ procurement organization. After the removal of the peripancreatic tissue and all visible vascular structures, it is chopped into 1 cm3 pieces. After decellularization, the cubes are lyophilized, cryomilled and gamma irradiated for sterilization. The powder is digested with pepsin-HCl for 48 hours at room temperature and neutralized with 0.1N NaOH and 10X PBS to obtain a pH of 7.4 at 4° C. This solution can be incubated at 37° C. for 1 hour for hydrogel formation, as described by Freytes et al. or can be further centrifuged and the supernatant can undergo another series of lyophilisation and cryomilling to produce the soluble ECM powder. Testing of pancreatic ECM with cells: We hypothesize that culture conditions that more closely resemble the environment of native islets will significantly improve the yield and viability of insulin producing cells during in vitro culture. We will obtain human ESC-derived pancreatic progenitor (PP; Stage 4; PDX1+/NKX6.1+/C-peptide-) cells for preliminary experiments. These cells will be seeded onto the microwell array (static) at different densities—500, 1000, 2000 and 4000 cells/spheroid and allowed to form compact microtissues over 3 days. The cell density that yields spheroids ˜150 1 μm in diameter (phase contrast images; ImageJ) will be used for further experiments. Experiments will also be conducted to optimize the overlaying of microwell arrays (containing spheroids) with the hydrogel. Once standardized, the protocols along with the plates (static and dynamic) and the ECM will be shared for subsequent experiments. The effect of three parameters: soluble ECM concentration, ECM hydrogel concentration and the flow rate of medium on cell viability (Live/Dead staining with Calcien AM & EthDII) and the yield of NKX6.1+/C-Peptide+cells (flow cytometry) in the ILCs and will be determined at the end of stage 7 of differentiation.

Testing of soluble ECM (3D static): Once compact spheroids have formed (day 3; day 0 of differentiation), three concentrations (0.05, 0.15 and 0.45 mg/ml) of the soluble ECM will be evaluated for their effect on the differentiation of spheroids. Concentrations>0.5 mg/ml were found to negatively affect the viability of cells in preliminary experiments. The ECM will be used as an additive in conjunction with the regular differentiation medium that has been well established. Medium will be changed every four days for ten to twenty days of differentiation (end of stage 6 and 7, respectively).

Testing of ECM hydrogel (3D static): Once compact spheroids have formed, the microwell array will be overlaid with the ECM solution and allowed to polymerize at 37° C. for 1 hour. Three concentrations of the hydrogel (3, 5 and 8 mg/ml) will be tested for their differentiation potential. Testing of combination (3D static): The best performing ECM concentrations (both soluble and hydrogel) from the previous experiments would be combined to determine if it is better than using them individually.

3D dynamic culture: The static condition resulting in the highest yield of NKX6.1+/C-peptide+ cells will be used for dynamic experiments. As the plate has an open assembly, the flow rate is dependent on the hydrostatic pressure, which is exerted by the height of the medium column. Three different heights (6, 7.5 and 9 mm) will be used to obtain increasing flow rates and evaluated for their effect on the differentiation of the spheroids. The feed rate of the pump will be determined to maintain a constant medium height, thus a constant flow rate. The flow rate is also dependent upon the hydraulic resistance offered by the transwell membrane, therefore a bigger pore size (5 or 8 μm) can be used to reduce the resistance and increase the flowrate of the system. Based on preliminary experiments, we assume that once fully hydrated, the hydrogel should offer minimal resistance and allow the medium to easily percolate through it.

Dynamic GSIS: Briefly, perifusing buffer (Krebs Ringer; KRB) at 37° C. with selected glucose (low; 2 mM, high; 16.7 mM) or KCl (30 mM) concentrations will be circulated through the plate at a rate of ˜100 μl/min. After 60 min of washing with the low glucose solution, ILCs will be stimulated with the following sequence: 5 min low, 20 min high glucose, 15 min low glucose and 10 min of KCl. Samples (100 μl) will be collected every two minutes from the outlet tubing. The plate and the buffers will be kept at 37° C. while the sample collection plate will be at 4° C. Insulin concentrations will be determined with commercially available ELISA kits.

Real-time fluorescence imaging: Imaging experiments for calcium dynamics and mitochondrial potential will be performed according to the protocol established in our laboratory. Briefly, ILCs will incubated with 5 μM Fura-2/AM (calcium indicator) and 2.5 μM Rhodamine 123 (Rh123, mitochondrial potential indicator) in KRB with 2 mM glucose for 30 min at 37° C. and the plate will be mounted on a confocal microscope. The plate will then be perfused with KRB with 2 mM glucose at 37° C. for 10 min to wash the ILCs. KRB containing 16.7 mM of glucose and 30 mM KCl will be administered for 15 min and 10 min, respectively, and simultaneously observed with a 4-20x (10-1 ILCs in field of view, respectively) objective. Dual-wavelength Fura-2 will be excited ratiometrically at 340 and 380 nm, and changes in [Ca2+] expressed as F340/F380 (%). Rh123 will be excited at 490 nm±10, and emission will be measured at 530 nm±10. Glucose-induced hyperpolarization of the mitochondrial membrane causes uptake of Rh123 resulting in decreased Rh123 fluorescence via quenching. Excitation and emission wavelengths will be controlled by means of suitable filters and dichroic.

### Example 2

Kidney disease is a debilitating condition affecting millions of Americans and leading to billions of dollars in healthcare costs. A significant impediment to the development of cell-based therapies for End Stage Renal Disease (ESRD) is the inability to generate or sustain mature human kidney organoids in culture. Recent studies have indicated that differentiation in 3D, signals from extracellular matrix (ECM) scaffolds and incorporation of fluid flow could have a positive impact on stem cell-derived organoid maturation. However, the controlled presentation of these physiological and extracellular matrix stimuli, their interplay and their combined roles in regulating differentiation, maturation and function of kidney organoids have not been demonstrated. Therefore, the overall objective of the proposed work is to define and optimize the synergistic effect of the different microenvironmental factors in guiding the differentiation of human induced pluripotent stem cell (hiPSC)—derived nephron progenitor (NP) cells towards a mature and functional kidney organoid. The ultimate goals of this proposal are threefold: 1) engineer an open, long-term culture system that is capable of providing robust control of the fluidic, biophysical and biochemical cellular microenvironment and allows for the assessment of multiple phenotypic and functional readouts; 2) utilize this innovative platform to systematically define and optimize critical factors capable of positively effecting the differentiation and maturation of organoids from human nephron progenitor cells; and 3) assess the impact of the optimal physiomimetic 3D niche on the function of organoids. The data produced in this study will demonstrate whether the application of these techniques can overcome the current challenges in this field. Finally, although the proposed study focuses on kidney organoid biomanufacturing from progenitor cells as an alternative approach for the treatment of ESRD, the tools and technologies developed herein are versatile and translatable to other tissues and cells. Hence, this investigation offers broad applicability to tissue engineering as a whole, especially to organ-on-a-chip and 3D bioprinting.

The limited availability of human organ donors renders kidney transplantation unlikely to provide a cure for End Stage Renal Disease (ESRD). Consequently, there is a significant interest in alternative cellular sources for kidney regeneration and research efforts have focused intensively on generating renal organoids from stem cells. However, the major barrier is the considerably low efficiency of the differentiation process and the inability to obtain mature kidney organoids in culture. To date, most studies have indicated that kidney organoids resemble the morphological and molecular signature of trimester 1 fetal kidneys and undergo dedifferentiation as well as enrichment of stromal and off-target cell populations with longer periods in culture. Even though differentiation in 3D has been more efficient than 2D, the inherent limitations of the culture platform might be a reason for such observations. Shortcomings, such as the absence of physiological fluid flow and lack of control on organoid size, resulting in larger constructs suffering from oxygen diffusion limitations, need to be addressed. Incorporation of perfusion has been shown to improve the maturation and function of microtissues, by providing an adequate nutrient and oxygen supply and well as mechanical cues through fluid shear stress (FSS). Furthermore, decellularized kidney extracellular matrix (ECM)—derived biomaterials could also provide a suitable platform for promoting the survival and maturation of renal cells. Taken together, the use of a culture platform that enables the formation of homogenously sized 3D microtissues and allows for continuous medium perfusion combined with ECM-derived biomaterials with preserved molecular integrity and minor constituents, would lead to advances in the field.

The microwell platform would enable the formation and guided differentiation of homogenously sized kidney organoids from human embryonic stem cell (hiPSC)—derived nephron progenitor (NP; Day 7 of differentiation from hiPSC for mesoderm induction; SIX2+SALL1+WT1+PAX2+) cells, while preventing their agglomeration, which could lead to diffusion limitations. The platform geometry enables direct flow of medium (through the microwells) rather than tangential (across; FIG. 1a, b), eliminating the possibility of inadequate medium exchange associated with traditional microwell-based devices. The plate was fabricated using rapid prototyping techniques, like laser machining and hot embossing, using non-absorbent and inert materials (polystyrene; PS & acrylic; PMMA). The platform is compatible with standard imaging platforms for time-resolved assessment of cellular readouts, and rapid assessment of organoid functionality, such as albumin uptake. The design being an open-well plate, rather than a closed organ-on-chip, would enable manual access for cell seeding, sampling and recovery of organoids for analysis or other applications like 3D bioprinting and implantation.

NP cells (day 7 of differentiation from hiPSC) are transferred from monolayers to either transwell filters (air-medium interface) or non-adherent culture platforms, resulting in cluster formation. As there is no physical constraint on size and separation, they can grow over time or agglomerate, which can result in diffusion limitations. Microwell arrays that physically restrict spheroid size can be used for achieving a defined and homogenous size. They can also be incorporated in microfluidic devices to sequester organoids in individual microwells, provide control over fluid flow and test them a dynamic manner. However, there are several disadvantages associated with conventional microfluidic devices. Medium flow across the face of microwell (tangential) can be inadequate in providing complete medium exchange, especially for deeper microwells. This was shown in a study, where devices with pancreatic islets trapped in cup shaped nozzles (open on both ends) stimulated intracellular flow, resulting in enhanced β-cell and endothelial preservation. Moreover, permanent bonding of devices can make cell loading and recovery difficult. Fabrication requires cleanrooms for soft lithography, and the fabrication material (polydimethylsiloxane; PDMS) is unsuitable due to absorption of hydrophobic reagents and leaching of small molecules. Finally, such devices might be suited for testing but cannot be easily scaled up for biomanufacturing organoids in physiomimetic conditions, discouraging their widespread adoption.

NP cells (SIX2+SALL1+WT1+PAX2+) would be seeded in the microwell platform to enable the formation of homogenously sized spheroids. These would be further differentiated either in the presence of soluble human kidney ECM, embedded in ECM hydrogel, or a combination of both, and exposed to different flow rates. We hypothesize that the combination of microenvironmental cues from the ECM and medium flow will result in a higher yield and maturation of kidney specific cells, compared to control conditions. We will determine which condition (or combination) will have the most effect on the enrichment and maturation of kidney organoids.

Kidney decellularization and ECM preparation: Briefly, human kidneys from disease free organ donors will be obtained from the local organ procurement organization. After the removal of the perinephric tissue and all visible vascular structures, it will be chopped into 1 cm3 pieces. After decellularization, the cubes will be lyophilized, cryomilled and gamma irradiated for sterilization. The powder will then be digested with pepsin-HCl for 48 hours at room temperature and neutralized with 0.1N NaOH and 10X PBS to obtain a pH of 7.4 at 4° C. This solution can be incubated at 37° C. for 1 hour for hydrogel formation, or can be further centrifuged and the supernatant can undergo another series of lyophilisation and cryomilling to produce the soluble ECM powder. Testing of kidney ECM with cells: We hypothesize that culture conditions that more closely resemble the environment of native kidneys will significantly improve the yield, viability and maturation of renal organoids in in vitro culture. We will receive human ESC-derived nephron progenitor (NP; Day 7 of differentiation from hiPSC for mesoderm induction; SIX2+SALL1+WT1+PAX2+) cells for preliminary experiments. These cells will be seeded onto the microwell array (static) at different densities—2000, 4000 and 8000 cells/spheroid and allowed to form compact microtissues over 3 days. The cell density that yields spheroids ˜300 μm in diameter (phase contrast images; ImageJ) will be used for further experiments. This particular size has been chosen as kidney organoids <200 μm were shown to have a low abundance of tubular structures, while those >700 μm showed presence of necrotic cores. Experiments will also be conducted to optimize the overlaying of microwell arrays (containing spheroids) with the hydrogel. Once standardized, the protocols along with the plates (static and dynamic) and the ECM will be used for subsequent experiments. The effect of three parameters: soluble ECM concentration, ECM hydrogel concentration and the flow rate of medium on cell viability (Live/Dead staining with Calcien AM & EthDII), maturation (Western blotting; WB) and the percentage yield of specific kidney cells (Flow Cytometry) will be determined at different stages of differentiation (Days 5, 10, 15 and 20). Expression of early markers of kidney commitment (EYA1, SIX1, and SIX2) and maturation markers for podocytes (NPHS1, NPHS2, and SYNPO), proximal tubule (SLC3A1, SLC6A13 and CUBN), distal tubule (SLC12A3), ureteric bud (Wnt9b), collecting duct (SPINK1), endothelial cells (CD31) and, a putative marker for stromal cells (FOXD1) will be analyzed. These markers are chosen based on recently published transcriptomic literature investigating the maturation stages of stem cell-derived kidney organoids. Further, WB will also be performed for the quantification of Collagen α-3(IV), α-4(IV) and α-5(IV) expressed by podocytes. Testing of soluble ECM (3D static): Once compact spheroids have formed, three concentrations (0.05, 0.15 and 0.45 mg/ml) of the soluble ECM will be evaluated for their effect on the differentiation of spheroids. Concentrations >0.5 mg/ml were found to negatively affect the viability of cells in preliminary experiments. The ECM will be used as an additive in conjunction with the regular differentiation medium that has been well established. Medium will be changed every two days for twenty (7+20) days of differentiation.

Testing of ECM hydrogel (3D static): Once compact spheroids have formed, the microwell array will be overlaid with the ECM solution and allowed to polymerize at 37° C. for 1 hour. Three concentrations of the hydrogel (3, 5 and 8 mg/ml) will be tested for their differentiation potential. During pilot experiments we observed that concentrations <3 mg/ml often do not polymerize completely or form fragile gels that might not be able to withstand fluid flow, while solutions >8 mg/ml can be too viscous, preventing the solution from entering the microwells and encapsulating the spheroids.

Testing of combination (3D static): The best performing ECM concentrations (both soluble and hydrogel) from the previous experiments would be combined to determine if it is better than using them individually.

3D dynamic culture: The static condition resulting in the maturation and highest yield of kidney specific cells will be used for dynamic experiments. As the plate has an open assembly, the flow rate is dependent on the hydrostatic pressure, which is exerted by the height of the medium column. Three different heights (6, 7.5 and 9 mm) will be used to obtain increasing flow rates and evaluated for their effect on the differentiation of the spheroids. The feed rate of the pump will be determined to maintain a constant medium height, thus a constant flow rate. Based on preliminary experiments, we assume that once fully hydrated, the hydrogel should offer minimal resistance and allow the medium to easily percolate through it.

To investigate the structural segmentation and function of mature kidney organoids, TEM and fluorescence-based albumin uptake assay, respectively, will be utilized.

Transmission Electron Microscopy (TEM) will be performed using the protocol that is well established in our laboratory. Ultrathin sections of 70-90 nm thickness will be sliced using a Ultramicrotome, collected onto 200-mesh copper grids and co-stained with uranyl acetate and lead citrate. TEM will be used for the detection of podocytes possessing foot processes, joined by slit diaphragm-like structures and urinary spaces under the foot processes. Further, immunogold labeling would be used for detecting nephrin and podocin in the slit diaphragms. While not wishing to be bound to any particular theory, we believe the mature organoids to have a trilaminar glomerular basement membrane (GBM) as opposed to a less-mature GBM double layered with two lamina rarae. The presence of microvilli and cilia in proximal tubules is also believed to be present.

Real-time fluorescence imaging (Albumin Uptake Assay): Imaging experiments for albumin uptake will be performed according to the protocol established in Sedrakyan, S., Da Sacco, S., Milanesi, A., Shiri, L., Petrosyan, A., Varimezova, R. & Perin, L. (2012). Injection of amniotic fluid stem cells delays progression of renal fibrosis. Journal of the American Society of Nephrology, 23(4), 661-673. Briefly, organoids will be pre-incubated for 60 min in Ringer solution, then exposed to 1 mg/ml Fluorescein isothiocyanate conjugated human serum albumin (FITC-HSA) in Ringer solution for 60 min at either 4° C. or 37° C. DiI (red) will be used for counterstaining live cells. Images will be acquired every 30 minutes with 20X/0.4 objective. The concentration of albumin in the medium will be quantified by ELISA.

While not wishing to be bound to any particular theory, we believe that the combination of enriched human ECM-derived biomaterials and the microwell perfusion plate will recapitulate the renal microenvironment, which would result in improved maturation, yield and functionality of cells. Specifically, we believe kidney organoids cultured in the presence of ECM and fluid flow to exhibit cell-specific maturation markers (WB quantification), higher percentage of kidney specific cells as well as a decrease in off-target population (Flow cytometry), compared to control conditions. The structural segmentation and ultrastructure (TEM; immunogold) associated with maturation is also believed to be present.

Organoid size, hydrogel composition and the flow rate may be altered. It is well known that organoid size can have an effect on the phenotype and function of cells. The microwell diameter can be increased to ˜700 enabling formation of larger organoids (300-500 μm). Secondly, the hydrogel and the soluble ECM are derived from whole human kidneys. However, ECM from different parts of the kidney can be rich in different biochemical factors, which can effect cell behavior. Therefore, ECM derived purely from the cortex or medulla or their different combinations can be used to determine their effect on organoid maturation and function. Thirdly, the flow rate is not only dependent on the hydrostatic pressure but also upon the hydraulic resistance offered by the transwell membrane, therefore a bigger pore size (5 or 8 instead of 3 μm) can be used to reduce the resistance and increase the flowrate of the system.

### Example 3

Herein, we propose to engineer a physiomimetic 3D pancreatic niche by combining a novel through-pore microwell perfusion plate and human pancreatic dECM produced using our detergent-free decellularization protocol. This platform would provide intimate control over the cellular microenvironment and the effect of various soluble and physiological factors on the differentiation and functional maturation of ILCs from human induced pluripotent stem cell (hIPSC)—derived pancreatic progenitor (PP) cells would be clearly defined.

Although, commercial entities have claimed industrial-scale manufacturing of insulin producing cells for transplantation applications, yet there is a need for platform and protocol optimization for reproducibly manufacturing and testing of functional ILCs at the laboratory level. The main objective here is to investigate the feasibility of a platform that can lead to the streamlining and semi-automation of the differentiation process and can be used for manufacturing as well as dynamic testing of ILCs at a research-scale, especially in laboratories that do not have access to commercial perifusion systems. The modular data generated from this study will create new knowledge in the field of pancreatic tissue engineering, as well as provide novel materials and platforms for mechanistic studies of islet biology in islet-on-a-chip, bioreactor and other tissue engineering platforms.

Type 1 diabetes (T1D; juvenile-onset diabetes) is an autoimmune disease resulting from the destruction of insulin-producing beta cells by one's own immune system. The use of islet transplantation to provide a replacement for the lost insulin-producing cells has proven to be an effective therapy, resulting in restoration of insulin secretion and of glucose homeostasis, and preventing complications associated with T1D. Therefore, extensive efforts have been directed towards generating functional β cells or islet-like clusters (ILCs) from inexhaustible resources like human induced pluripotent stem cells (hIPSC). However, a major barrier in generating insulin producing cells is the considerably low yield of the differentiation process and the inability to sustain mature β cells in culture. This inability to obtain islet maturity and maintain function in culture underscores the significance of bioengineering a physiomimetic pancreatic niche. Here, we have identified three factors that should be synergistically employed to engineer a physiologically relevant microenvironment for long-term culture, functional maturation and maintenance of ILCs; 1) use of a microwell array to produce homogenously sized ILCs and prevent agglomeration, 2) incorporation of perfusion to allow for continuous medium exchange, provide mechanical cues and removal of debris and 3) human pancreatic dECM to provide more relevant biochemical cues. The rational for including these factors in the platform is described below.

At Stage 5 of differentiation, pancreatic progenitor (PP) cells are transferred to non-adherent growth platforms, resulting in cluster formation. As there is no physical constraint on size and separation, clusters can grow over time or agglomerate, leading to diffusion limitations. Microwell arrays that physically restrict spheroid size can be used for achieving a defined and homogenous size. However, long-term culture and differentiation of clusters in microwell arrays is generally not possible due to accumulation of cell debris, which is detrimental to cell viability and function. Therefore, clusters are transferred to standard culture platforms for continued differentiation, where they can again grow in size and agglomerate. Moreover, frequent medium changes and functionality testing (Glucose stimulated insulin secretion; GSIS) can present logistical difficulties, often requiring multiple centrifugation and resuspension steps to prevent loss of clusters during the process. Spinner flasks can also be used in order to provide a dynamic environment and prevent agglomeration of clusters but being low throughput renders them unsuitable for parallel testing of multiple growth and differentiation factors.

Microwell arrays have also been incorporated in microfluidic devices to sequester spheroids in individual microwells, provide control over fluid flow and test islets a dynamic manner. Although, such devices might be suited for testing and characterization of islets but cannot be easily adapted for long-term culture and differentiation of ILCs. Moreover, there are several other disadvantages associated with conventional microfluidic devices. Medium flow across the face of microwell (tangential) can be inadequate in providing complete medium exchange, especially for deeper microwells. It has been shown that devices with islets trapped in cup shaped nozzles (open on both ends) stimulated intracellular flow, resulting in enhanced β-cell preservation. Additionally, fabrication of these devices generally requires cleanrooms and specialized equipment for soft lithography. The fabrication material (polydimethylsiloxane; PDMS) is unsuitable due to absorption of hydrophobic reagents, which decreases their intended concentration, and leaching of small molecules like endocrine disruptor cyclosilane into the medium. To overcome this limitation, non-porous thermoplastics like Polymethyl methacrylate (PMMA) have been used for fabrication. However, they are not conducive to gas exchange and a sealed PMMA microfluidic chip might not be suitable for hypoxia-sensitive islets. Furthermore, permanent bonding or sealing of devices can also make cell loading and recovery for downstream analysis difficult, discouraging their widespread adoption. Therefore, the development of an open culture platform that restricts cluster size, allows removal of cell debris, enables direct perfusion flow of medium, high-resolution real-time imaging and easier retrievability of clusters would be well-suited for manufacturing (formation, differentiation, maturation, maintenance and testing) of ILCs.

Taken together, there is a need for an open, long-term culture system that provides robust control on the fluidic, biophysical and biochemical microenvironment and allows for multiple characterization and functional readouts in order to optimize critical factors that positively affect the differentiation, maturation and function of ILCs.

Our approach is innovative for at least three reasons: 1) a 3D microwell-based perfusion plate that supports the formation, long-term culture, dynamic testing and simultaneous live-cell imaging of islet-like clusters (ILCs); 2) a “gentler” pancreas decellularization method that preserves essential compounds (Glycosaminoglycans and other small molecules), and the subsequent potent solubilized dECM powder that can be used as a medium additive; 3) combination of the two that provides the essential spatial, biochemical and biophysical cues in a dynamic milieu, to create a physiomimetic niche for manufacturing ILCs.

As described herein, we have recently developed an open cell culture platform that combines the advantages of microfluidic chips, bioreactors and microwell arrays. This microwell perfusion platform would enable the formation and guided differentiation of homogenously sized ILCs from human embryonic stem cell (hESC)—derived pancreatic progenitor (PP) cells, while preventing their agglomeration, which could lead to diffusion limitations. The platform geometry enables direct flow of medium (through the microwells) rather than tangential (see, e.g., FIG. 1), eliminating the possibility of inadequate medium exchange associated with traditional microwell-based devices. Such direct fluid flow, along with the combination of the through-pore microwell and transwell membranes would also allow for the removal of cell debris and prevent its accumulation in the microwells during differentiation. Perfusion would also enable easier change of growth factors and medium during different stages of differentiation and maturation and create bioreactor-like conditions but at a higher throughput. The plate was fabricated using rapid prototyping techniques, like laser machining and hot embossing, using non-absorbent and inert materials (polystyrene; PS & acrylic; PMMA) and has a standard multiwell plate footprint. The platform is compatible with standard imaging platforms for time-resolved assessment of cellular readouts, and rapid assessment of islet functionality, such as in-plate dynamic GSIS. Moreover, the plate eliminates the need for manually counting and picking of clusters for performing GSIS and same ILCs can be tested multiple times over the differentiation period to establish a time-profile. The design being an open-well plate, rather than a closed islet-on-chip, would provide optimum oxygen exchange and allow manual access for cell seeding, sampling and recovery of ILCs for analysis or other applications like 3D bioprinting and implantation. As the fluidic connections are through the lid rather than the body of the plate, the system can be easily disconnected from the fluidics by replacing it with a regular lid for easier routine microscopy, eliminating the need to transfer the fluidic setup (pump and tubing).

These methods have never been combined to develop a single platform for manufacturing (formation, differentiation, maturation, maintenance and testing) of ILCs from hIPSC-derived PP cells. Moreover, the microwell perfusion plate could provide a worthwhile alternative to both bioreactor-based maturation of ILCs and to perform perifusion assay (GSIS) for research groups that lack access or technical skill. Pancreatic dECM has been proposed here as a means of providing physiologically relevant biochemical factors to the ILCs. However, following a similar template, the microwell perfusion plate could also be used as a medium throughput screening platform (ILCs-on-a-chip) for parallel testing of several other biochemical factors, individually or in combination. Through this research, we will demonstrate proof of concept and open doors to innovations in the field of islet biology and eventually diabetes treatment. This system can also be adopted for the maintenance of human islets, maturation of neonatal porcine islets as well as other organ systems.

More points: 1) Mini-bioreactor for stem cell derived organoids; 2) One platform/device for biomanufacturing—formation, differentiation, maturation, maintenance and testing of organoids—streamline and semi-automate; 3) Continuous in-plate monitoring with dynamic assays and real-time imaging—generate a time profile for organoid characteristics (phenotype, function etc); 4) parallel testing of multiple conditions—medium throughput.

The plate exists in two configurations —static and perfusion—with the through-pore microwell membrane (Polystyrene; PS) bonded onto a porous transparent transwell membrane (Polycarbonate; PC) being central to both. The body was fabricated by laser micromachining multiple PMMA layers with integrated fluidic channels and the PS and PC membranes were sandwiched between them (see, e.g., FIGS. 5, 23) The whole assembly was then bonded onto a well of a standard six-well plate (see, e.g., FIGS. 9, 26). The organoid culture well can accommodate 200 ILCs (one in each microwell) in order to produce enough insulin to be in the linear detection range of the Insulin ELISA. The plate offers control over parameters such as number of microwells per well and the flow rate, by changing the height of the cell culture well (hydrostatic head) and pore size of the PC transwell membrane (5, 8 or 10 μm). The bottom layer of outlet channel is laser cut directly on the 6 well plate and bonded with a #1 coverslip for reducing the height of ILCs from the microscope lens and providing optical accessibility for improved high-resolution imaging.

The key design difference between the static and perfusion plates is the medium residence volume, governed by the shape of channels. It has been maximized in the static plate (˜2 ml) to reduce the frequency of medium changes, while reduced in dynamic (˜600 ul) to minimize the dead volume. The lid for the perfusion plate has metal couplers (27 gauge) that connect the inlet and outlet to a peristaltic pump via silicone tubing. The feed rate of the pump can be attuned to maintain a constant medium height, thus a constant flow rate. The fluidic circuit of the perfusion plate has also been fitted with switches for inflow of low or high glucose solutions for performing dynamic GSIS. This platform would result in high cluster number per unit area, minimize loss of clusters during medium change, while allowing for high retrievability of clusters, removal of cell debris, adequate oxygen exchange and separation of apical and basal medium as well as high resolution imaging and in-plate functional testing (static or dynamic GSIS) of ILCs.

Our strategy is to combine a novel microwell-based perfusion plate with human pancreatic dECM to develop a physiomimetic 3D niche that should result in improved yield, viability and function of ILCs.

The liquid column (hydrostatic head; height or depth of cell culture well) will dictate the hydrostatic pressure on the membrane, which needs to be high enough to drive out cell debris during medium change. The current depth of the cell culture well is 3 mm, which can be increased by incorporating additional PMMA layers. Different depths (3, 6, 7.5 and 9 mm) will be tested and the minimum depth that results in the removal of cell debris during medium change will be determined and used for further experiments.

Access to commercial perifusion systems needed to conduct dynamic GSIS is limited to a few research institutes dedicated to diabetes research. Therefore, our goal is to adapt the microwell perfusion plate to perform dynamic GSIS with fluidic systems available in regular laboratories, thereby making the technology more accessible. We will investigate multiparameteric real-time cellular response to glucose challenge by testing the feasibility of in-plate dynamic GSIS and simultaneous live cell imaging.

### Example 4

Type 1 diabetes (T1D; juvenile-onset diabetes)) is an autoimmune disease resulting from the destruction of the insulin-producing beta cells by one's own immune system. The use of islet transplantation to provide a replacement for the lost insulin-producing cells has proven to be an effective therapy, resulting in restoration of insulin secretion and glucose homeostasis, and preventing complications associated with T1D. Therefore, extensive efforts have been directed towards generating functional β cells or islet-like clusters (ILCs) from inexhaustible resources like human induced pluripotent stem cells (hIPSC). However, a major barrier in generating insulin-producing cells is the considerably low yield of the differentiation process and the inability to sustain mature β cells in culture. This inability to obtain β cell maturity and maintain function in culture underscores the significance of bioengineering a physiomimetic pancreatic niche. Here, we have identified three design components that should be incorporated in the culture system to engineer a physiologically relevant microenvironment for long-term culture, functional maturation and maintenance of ILCs: 1) the use of a microwell array to produce homogenously sized ILCs and prevent agglomeration, 2) the incorporation of perfusion to allow for continuous medium exchange, provide mechanical cues and removal of debris and 3) human pancreatic dECM to provide more relevant biochemical cues. The rationale for including these three factors in the platform is described below.

Conventional culture platforms. In conventional cultures, hPSCs are aggregated and cultured under suspension culture, and differentiated following sequential induction through Definitive Endoderm (DE), pancreatic progenitor (PP 1, 2) and endocrine progenitor (EN) stages. At the EN stage, the clusters are dissociated, sorted for monohormonal □-like cells, re-aggregated and matured further under suspension culture. Such suspension culture of the iPSCs, however, often results in uncontrolled cluster formation, which compromises mature function. As there is no physical constraint on size and separation, clusters tend to grow over time or agglomerate, leading to diffusion limitations. Microwell arrays that physically restrict spheroid size can be used for achieving and retaining defined and homogenous cluster size. However, long-term culture and differentiation of clusters in microwell arrays is generally not possible due to accumulation of cell debris, which is detrimental to cell viability and function. Therefore, clusters are transferred to standard culture platforms for continued differentiation, where they can grow in size and agglomerate. Moreover, frequent medium changes and functionality testing (glucose stimulated insulin secretion: GSIS) can present logistical difficulties, often requiring multiple centrifugation and resuspension steps to prevent loss of clusters during the process. Spinner flasks can also be used in order to provide a dynamic environment and prevent agglomeration of clusters but being low throughput renders them unsuitable for parallel testing of the effects of multiple growth and differentiation factors on β cell maturation and functionality.

Microfluidic Devices. Microwell arrays have also been incorporated in microfluidic devices to sequester spheroids in individual microwells, provide control over fluid flow and test islets in a dynamic manner. Although, such devices might be suited for testing and characterization of islets, they cannot be easily adapted for long-term culture, differentiation and maturation of ILCs. Moreover, there are several disadvantages associated with conventional microfluidic devices. Medium flow across the face of microwell (tangential) can be inadequate in providing complete medium exchange, especially for deeper microwells. It has been shown that devices with islets trapped in cup shaped nozzles (open on both ends) that enabled direct intracellular flow, resulted in enhanced β-cell preservation. Additionally, fabrication of these devices generally requires cleanrooms and specialized equipment for soft lithography. The fabrication material (polydimethylsiloxane; PDMS) is unsuitable due to protein absorption, which decreases their intended concentration, and leaching of small molecules like endocrine disruptor cyclosilane into the medium. To overcome this limitation, non-porous thermoplastics like Polydimethyl methacrylate (PMMA) have been used for fabrication. However, they are not conducive to gas exchange and a sealed PMMA microfluidic chip might not be suitable for hypoxia-sensitive islets. Furthermore, permanent bonding or sealing of devices can also make cell loading and recovery for downstream analysis difficult, discouraging their widespread adoption. Recently, it was shown that culture of IPSC-derived kidney organoids under perfusion conditions in a fluidic chip can result in enhanced phenotypic and functional maturation of the organoids, compared to static conditions. However, the effect of perfusion and fluid flow on the maturation of ILCs has not been investigated yet. Therefore, the development of an open culture platform that restricts cluster size, allows removal of cell debris, enables direct perfusion of culture media and high-resolution real-time imaging with easier retrievability of clusters would be well-suited for manufacturing (formation, differentiation, maturation, maintenance and testing) of ILCs.

Taken together, there is a need for an open, long-term culture system that provides robust control on the fluidic, biophysical and biochemical microenvironment and allows for multiple characterization and functional readouts in order to optimize critical factors that positively affect the differentiation, maturation and function of ILCs.

Our approach is innovative for three reasons: 1) a 3D microwell-based perfusion plate that supports the formation, long-term culture, dynamic testing and simultaneous live-cell imaging of ILCs; 2) a “gentler” pancreas decellularization method that preserves essential compounds (glycosaminoglycans and other small molecules), generating a potent solubilized dECM powder (lyophilized) that can be used as a culture media additive; 3) the combination of the two that provides the critical spatial, biochemical and biophysical cues in a dynamic milieu, to create a physiomimetic niche for manufacturing ILCs.

We have recently developed an open cell culture platform that combines the advantages of microfluidic chips, bioreactors and microwell arrays. This microwell perfusion platform will enable the formation and guided differentiation of homogenously sized ILCs from hiPSC-derived EN (Endocrine Progenitor) cells, while preventing their agglomeration, which could lead to diffusion limitations. The platform geometry enables direct flow of medium through the microwells rather than tangential (across; FIG. 1), eliminating the possibility of inadequate medium exchange associated with traditional microwell-based devices. Such direct fluid flow, along with the combination of the through-pore microwell and transwell membranes would also allow for the removal of cell debris and prevent its accumulation in the microwells during hiPSC differentiation and maturation. Perfusion will also enable easier change of growth factors and medium during the different stages of differentiation and maturation and create bioreactor-like conditions but with a higher throughput. The plate was fabricated using rapid prototyping techniques, like laser machining and hot embossing, using non-absorbent and inert materials (polystyrene; PS & acrylic; PMMA) and has a standard multiwell plate footprint. The platform is compatible with standard imaging platforms for time-resolved assessment of cellular readouts, and rapid assessment of islet functionality, such as, in-plate dynamic GSIS. Moreover, the plate eliminates the need for manually counting and picking of clusters for performing GSIS and the same ILC aliquot can be tested multiple times over the differentiation period to establish a time-profile. The design being an open-well plate, rather than a closed microfluidic chip/device, would provide optimum oxygen exchange and allow manual access for cell seeding, sampling and recovery of ILCs for analysis or other applications, like 3D bioprinting and implantation. Moreover, as the fluidic connections are through the lid rather than the body of the plate, the system can be easily disconnected from the fluidics by replacing it with a regular lid for easier routine microscopy, eliminating the need to transfer the fluidic setup (pump and tubing).

These methods have never been combined to develop a single platform for manufacturing (formation, differentiation, maturation, maintenance and testing) of ILCs from hIPSC-derived EN cells. Moreover, the microwell perfusion plate could provide a worthwhile alternative to both bioreactor-based maturation of ILCs and to traditional perifusion systems for research groups that lack access to this equipment or technical skill. Pancreatic dECM has been proposed here as a means of providing physiologically-relevant biochemical factors to the ILCs. However, following a similar template, the microwell perfusion plate could also be used as a culture media high-throughput screening platform (ILCs-on-a-chip; Microphysiological System, MPS) for parallel testing of several other biochemical factors, individually or in combination. Here, we will conduct proof of concept experiments that will advance the field of islet biology and will validate an innovative combination platform to produce ILCs for beta cell replacement therapy in T1D. We envision that our platform can also be adopted for the long-term culture of human islets and for promoting maturation of neonatal porcine islets.

Fabrication of the Microwell Plate. The plate exists in two configurations —static and perfusion— with the through-pore microwell array (Polystyrene; PS) bonded onto a porous transparent transwell membrane (Polycarbonate; PC) being central to both. The body was fabricated by laser micromachining multiple PMMA layers with integrated fluidic channels and the PS and PC membranes were sandwiched between them. The whole assembly was then bonded onto a well of a standard six-well plate. The organoid culture can accommodate 200 ILCs (one in each microwell) in order to produce enough insulin to be in the linear detection range of the Insulin ELISA. The plate offers control over the number of microwells per well, by changing the diameter of the organoid culture well (CW), and also the flow rate, by changing the height of the cell culture well (hydrostatic head) and the pore size of the PC transwell membrane (3, 5, 8 or 10 μm). The bottom layer containing the outlet channel is laser cut directly on the 6 well plate and bonded with a #1 coverslip for optical accessibility and high-resolution imaging. The key design difference between the static and perfusion (dynamic) plate is the medium residence volume (MRV), governed by the shape of channels. The MRV has been maximized in the static plate (˜2 ml) to reduce the frequency of culture media changes, while it was reduced in the perfusion plate (˜600 ul) to minimize the dead volume and diminish the lag in glucose response. The fluidic circuit of the perfusion plate has also been fitted with switches (Idex #V-100D) for bubble-free inflow of low/high glucose solutions for performing dynamic GSIS. This platform will result in high cluster number/area, minimize loss of clusters during media refresh, while allowing for retrievability of clusters, removal of cell debris, adequate oxygen exchange and separation of apical and basal media, as well as high resolution imaging and capability of in-plate functional testing (static or dynamic GSIS) of ILCs.

We propose to combine a novel microwell-based perfusion plate with human pancreatic dECM into a physiomimetic 3D niche and test whether this platform will improve yield, viability and function of ILCs during long-term culture. We hypothesize that ILCs cultured in the through-pore microwell plate will exhibit significantly improved yield, viability and function of ILCs during in vitro culture.

Access to commercial perifusion systems for conducting dynamic GSIS is limited to a few diabetes-focused research institutes. Therefore, our goal is to adapt the microwell perfusion plate to perform dynamic GSIS with fluidic systems available in regular laboratories, thereby making the technology more accessible to the scientific community. We will investigate multiparameteric real-time cellular response to glucose challenge by testing the feasibility of in-plate dynamic GSIS and simultaneous live cell imaging.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1. A microwell perfusion plate system comprising:
a plate;
at least one well on the plate, each well comprising:
a porous membrane;
a through-pore microwell membrane having a top and a bottom with the bottom above and on the porous membrane, the microwell membrane comprising a plurality of microwells with a respective microwell configured to hold a 3D cell culture, wherein a respective microwell comprises a top opening at the top of the microwell membrane and a bottom opening at the bottom of the microwell membrane;
an inlet passageway in fluid communication with each top opening of the plurality of microwells and configured to deliver liquid medium to the plurality of microwells and the 3D cell cultures held therein;
an outlet passageway in fluid communication with each bottom opening of the plurality of microwells and configured to receive the liquid medium from the plurality of microwells; and
a cell culture well directly above the microwell membrane, wherein the cell culture well defines at least a portion of the inlet passageway.

2. The system of claim 1 wherein each well comprises a bottom outlet channel below the porous membrane and extending between a central portion of the well and an outer peripheral portion of the well, and wherein the bottom outlet channel defines at least a portion of the outlet passageway.

3. The system of claim 2 wherein the bottom outlet channel widens from the central portion of the well to the outer peripheral portion of the well.

4. The system of claim 2 wherein the bottom outlet channel has a constant width or narrows from the central portion of the well to the outer peripheral portion of the well.

5. The system of claim 2 wherein the bottom outlet channel is defined in the plate.

6. The system of claim 2 wherein:
each well comprises a body comprising at least one layer that is on the microwell membrane and/or the plate; and
the cell culture well is defined in a central portion of the body.

7. The system of claim 6 wherein the body is bonded to the plate.

8. The system of claim 6 wherein the body and/or the plate comprise PMMA.

9. The system of claim 6 wherein the body is on a first side of the plate, each well further comprising a glass coverslip on a second, opposite side of the plate below the bottom outlet channel.

10. The system of claim 6 further comprising an outlet medium reservoir optionally defined in an outer peripheral portion the body, the outlet medium reservoir in fluid communication with and positioned above the bottom outlet channel optionally at the outer peripheral portion of the well, wherein the outlet medium reservoir defines at least a portion of the outlet passageway.

11. The system of claim 10 wherein the outlet medium reservoir is arcuate and extends along a portion of the outer peripheral portion the body.

12. The system of claim 6 further comprising an inlet medium compartment defined in the body, the inlet medium compartment in fluid communication with and positioned above the cell culture well, wherein the inlet medium compartment defines at least a portion of the inlet passageway.

13. The system of claim 12 wherein:
the outlet medium reservoir is at a first side of the outer peripheral portion of the body; and
the inlet medium compartment extends between the outlet medium reservoir and a second, opposite side of the outer peripheral portion of the body.

14. The system of claim 12 wherein the body further comprises an inlet port member at the outer peripheral portion of the body, the inlet port member comprising an inlet port configured to receive a pipette tip such that the liquid medium is delivered to the inlet medium compartment.

15. The system of claim 14 wherein:
the body comprises first and second layers;
the cell culture well and a lower portion of the outer medium reservoir are defined in the first layer;
the inlet medium compartment and an intermediate or upper portion of the outlet medium reservoir are defined in the second layer; and
the inlet port member is on the second layer.

16. The system of claim 15 wherein the intermediate or upper portion of the outlet medium reservoir is an intermediate portion of the outlet medium reservoir, and wherein the body further comprises an upper portion of the outlet medium reservoir on the second layer and above the intermediate portion of the outlet medium reservoir.

17. The system of claim 12 wherein the inlet medium compartment diverges into first and second inlet fluid pathways at the outer peripheral portion of the body and the first and second inlet fluid pathways converge at the central portion of the body above the cell culture well.

18. The system of claim 12 or 17 wherein the body further comprises an inlet and outlet port member comprising an inlet port in fluid communication with and positioned above the inlet medium compartment and an outlet port in fluid communication with and positioned above the outlet medium reservoir.

19. The system of claim 18 wherein:
the body comprises first, second, and third layers;
the cell culture well and a lower portion of the outlet medium reservoir are defined in the first layer;
the inlet medium channel and an upper portion of the outlet medium reservoir are defined in the second layer; and
the inlet port and the outlet port are defined in the third layer.

20. The system of claim 6 wherein the body is monolithic.

21. The system of claim 18 further comprising a lid configured to be selectively installed over the second side of the plate, the lid comprising an inlet port and an outlet port for each well, the inlet port of the lid in fluid communication with the inlet port of the body and the outlet port of the lid in fluid communication with the outlet port of the body.

22. The system of claim 21 further comprising:
an inlet coupler in the inlet port of the lid;
an outlet coupler in the outlet port of the lid;
an inlet tube connected to the inlet coupler at a first end of the inlet tube;
an outlet tube connected to the outlet coupler at a first end of the outlet tube;
at least one pump with a second, opposite end of the inlet tube connected to the at least pump and a second, opposite end of the outlet tube connected to the at least one pump;
wherein the pump is configured to deliver medium to the body through the inlet tube and remove medium from the body through the outlet tube.

23. The system of claim 22 wherein:
the inlet coupler extends downwardly into the inlet port of the inlet and outlet port member; and
the outlet coupler extends downwardly into the outlet port of the inlet and outlet port member.

24. The system of claim 1 further comprising an insert configured to be selectively installed in a container held in a respective well, wherein:
the porous membrane and the through-pore microwell membrane are on the insert;
the cell culture well is on the insert and surrounds the porous membrane and the through-pore microwell membrane; and
the container defines at least a portion of the outlet passageway.

25. The system of claim 24 further comprising a lid configured to be selectively installed over the first side of the plate, the lid comprising an inlet port and an outlet port for each well, the inlet port of the lid in fluid communication with the cell culture well and the outlet port of the lid in fluid communication with the container.

26. The system of claim 25 further comprising:
an inlet coupler in the inlet port of the lid;
an outlet coupler in the outlet port of the lid;
an inlet tube connected to the inlet coupler at a first end of the inlet tube;
an outlet tube connected to the outlet coupler at a first end of the outlet tube;
at least one pump with a second, opposite end of the inlet tube connected to the at least pump and a second, opposite end of the outlet tube connected to the at least one pump;
wherein the pump is configured to deliver medium to the cell culture well through the inlet tube and remove medium from the container through the outlet tube.

27. The system of claim 26 wherein the inlet coupler extends downwardly into the cell culture well and the outlet coupler extends downwardly into the container.

28. The system of claim 1 wherein the at least one well comprises a plurality of wells.

29. The system of claim 1 wherein a respective microwell of the microwell membrane has a pyramidal shape.

30. The system of claim 1 wherein a respective microwell of the microwell membrane comprises sloped trapezoidal sidewalls and the top and bottom openings are square.

31. The system of claim 1 wherein a respective microwell of the microwell membrane comprises a curved sidewall, the top and bottom openings are circular and/or round, and the microwell has a hemispherical shape.

32. A method of culturing cells and/or preparing organoids, spheroids, microtissues, and/or cell clusters, the method comprising:
providing the system of claim 1; and
perfusing the liquid medium directly through the top opening, past and/or through the 3D cell culture, and then through the bottom opening of each microwell of the microwell membrane.

33. A method of culturing cells and/or preparing organoids, the method comprising:
providing a system comprising:
a plate comprising a plurality of wells;
a porous membrane in each well;
a microwell through-pore membrane directly above and on the porous membrane, the microwell membrane comprising a plurality of microwells, each microwell comprising at least one sidewall defining a top opening at a top of the microwell membrane and a bottom opening at a bottom of the microwell membrane, each microwell configured to hold a 3D cell culture;
a cell culture well directly above the microwell membrane; and
an outlet medium reservoir with at least a portion of the outlet medium reservoir directly below the porous membrane; and
directly perfusing liquid medium through the cell culture well, then through the top opening of each microwell, then past and/or through the 3D cell culture, then through the bottom opening of each microwell, and then through the outlet medium reservoir.
